SEQUENCE LISTING (21 sequences)

SEQ ID NO: 1 - protein, 578 aa, Homo sapiens
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 2 - protein, 577 aa, Homo sapiens
MLLLINVILTLWVSCANGQVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 3 - protein, 331 aa, Homo sapiens
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 4 - protein, 19 aa, Homo sapiens
MLLLINVILTLWVSCANGQ

SEQ ID NO: 5 - protein, 558 aa, Homo sapiens
EVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 6 - protein, 312 aa, Homo sapiens
EVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 7 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 8 - protein, 645 aa, Homo sapiens
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAA

SEQ ID NO: 9 - protein, 915 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 10 - DNA, 21 bp, Artificial Sequence
  Construct FHR4 #1 Position 46 siRNA target
gaatcacacttggtaactaat

SEQ ID NO: 11 - DNA, 21 bp, Artificial Sequence
  Construct FHR4 #1 Position 46 siRNA scrambled
gccaagaatgtacctattata

SEQ ID NO: 12 - DNA, 21 bp, Artificial Sequence
  Construct FHR4 #2 Position 835 siRNA target
ggtcaagagtcgagtaccagt

SEQ ID NO: 13 - DNA, 21 bp, Artificial Sequence
  Construct FHR4 #2 Position 835 siRNA scrambled (negative control)
gggtgaatcccgcatggtaaa

SEQ ID NO: 14 - DNA, 21 bp, Artificial Sequence
  Construct FHR4 #3 Position 1401 siRNA target
gaatgctacgatggatatgaa

SEQ ID NO: 15 - DNA, 21 bp, Artificial Sequence
  Construct FHR4 #3 Position 1401 siRNA scrambled (negative control)
gataaggtgagccattagata

SEQ ID NO: 16 - DNA, 21 bp, Artificial Sequence
  Construct FHR4 #4 Position 1658 siRNA target
ggaaccaccaagatgcataca

SEQ ID NO: 17 - DNA, 21 bp, Artificial Sequence
  Construct FHR4 #4 Position 1658 siRNA scrambled (negative control)
gcactcaagaagacgatacca

SEQ ID NO: 18 - DNA, 21 bp, Artificial Sequence
  Construct FHR4 #5 Position 1794 siRNA target
ggatataatgcgaatacatca

SEQ ID NO: 19 - DNA, 21 bp, Artificial Sequence
  Construct FHR4 #5 Position 1794 siRNA scrambled (negative control)
gcataagaatatacgttcaga

SEQ ID NO: 20 - DNA, 21 bp, Artificial Sequence
  Construct FHR4 #6 Position 2154 siRNA target
gcatattgtacagtataccta

SEQ ID NO: 21 - DNA, 21 bp, Artificial Sequence
  Construct FHR4 #6 Position 2154 siRNA scrambled (negative control)
gttgctatcgtctataaacaa

(19) United States
(12) Patent Application Publication
(10) Pub. No.: US 2021/0246507 A1
(43) Pub. Date: Aug. 12, 2021

(54) METHODS FOR ASSESSING MACULAR DEGENERATION

(71) Applicant: The University of Manchester, Manchester, Greater Manchester (GB)

(72) Inventors: Simon CLARK, Manchester, Greater Manchester (GB); Paul BISHOP, Manchester, Greater Manchester (GB)

(21) Appl. No.: 17/054,155

(22) PCT Filed: May 10, 2019
(86) PCT No.: PCT/EP2019/062048

§ 371 Date: Nov. 9, 2020

(30) Foreign Application Priority Data
May 10, 2018 (GB) 1807611.7
Mar. 1, 2019 (GB) 1902790.3

(51) Int. Cl. C12Q 1/6883

(52) U.S. Cl. CPC C12Q 1/6883; C12Q 2600/118; C12Q 2600/158

(57) ABSTRACT

Methods for determining whether a subject is at risk of developing a complement-related disorder are disclosed. Also disclosed are complement-targeted therapeutics for treating a complement-related disorder, in particular agents that decrease the level of FHR-4.

This application claims priority from GB1807611.7 filed 10 May 2018 and from GB1902790.3 filed 1 Mar. 2019, the contents and elements of which are herein incorporated by reference for all purposes.

### FIELD OF THE INVENTION

The present invention relates to the fields of molecular biology and medicine. More specifically, the present invention relates to methods for assessing risk of developing complement-driven disorders, and methods of treating said disorders.

### BACKGROUND

The complement system is a part of the innate immune system and performs major roles in the elimination of microbes, inflammatory processes, disposal of cellular debris and modulation of adaptive immunity (Ricklin D., Nat. Immunol. 2010, 11, 785-797). Complement is activated by the deposition onto a surface of protein C3b, a pro-inflammatory breakdown product of immune system protein C3. C3b associates with other proteins to form convertase enzyme complexes for activating and amplifying complement responses, and initiates an amplification loop of the complement cascade. This ultimately leads to cell/tissue destruction and a local inflammatory response. Consequently, deregulation and deficiencies, e.g. over-activation, of the complement system is implicated as a key driver of numerous inflammatory, autoimmune, neurodegenerative, and infectious disorders (McGeer P L et al., Neurobiol Aging. 2017, 52:12-22).

Macular degeneration, e.g. age-related macular degeneration (AMD), is believed to be caused, in part, by complement-mediated attack on ocular tissues. AMD is the leading cause of blindness in the developed world: currently responsible for 8.7% of all global blind registrations, it is estimated that 196 million people will be affected by 2020, increasing to 288 million by 2040 (Wong et al. Lancet Glob Heal (2014) 2:e106-16). AMD manifests as the progressive destruction of the macula, the central part of the retina at the back of the eye, leading to loss of central visual acuity. Early stages of the disease see morphological changes in the macula, including first the loss of blood vessels in the choriocapillaris (Whitmore et al., Prog Retin Eye Res (2015) 45:1-29); a layer of capillaries found in the choroid (a highly vascularized layer that supplies oxygen and nutrition to the outer retina). The choriocapillaris is separated from the metabolically active retinal pigment epithelium (RPE) by Bruch's membrane (BrM), a thin (2-4 μm), acellular, five-layered sheet of extracellular matrix. The BrM serves two major functions: the substratum of the RPE and a blood vessel wall. The structure and function of BrM is reviewed e.g. in Curcio and Johnson, Structure, Function and Pathology of Bruch's Membrane, In: Ryan et al. (2013), Retina, Vol. 1, Part 2: Basic Science and Translation to Therapy. 5th ed. London: Elsevier, pp 466-481, which is hereby incorporated by reference in its entirety.

The role of complement in AMD is reviewed, for example, by Zipfel et al. Chapter 2, in Lambris and Adamis (eds.), Inflammation and Retinal Disease: Complement Biology and Pathology, Advances in Experimental Medicine and Biology 703, Springer Science+Business Media, LLC (2010), which is hereby incorporated by reference in its entirety. The key characteristics of AMD are indicative of over-active complement, including cell/tissue destruction and a local inflammatory response. Hallmark lesions of early AMD, termed drusen, develop within BrM adjacent to the RPE layer (Bird et al, Surv Ophthalmol 1995, 39(5):367-374). Drusen are formed from the accumulation of lipids and cellular debris, and include a swathe of complement activation products (Anderson et al., Prog Retin Eye Res 2009, 29:95-112; Whitcup et al., Int J Inflam 2013, 1-10). The presence of drusen within BrM disrupts the flow of nutrients from the choroid across this extracellular matrix to the RPE cells, which leads to cell dysfunction and eventual death, leading to the loss of visual acuity.

Genetic alterations/variations are a major risk factor for AMD. Recently, 45 common single nucleotide polymorphisms (SNPs) and 7 rare variants across 34 genetic loci have been associated with this condition, explaining up to 34% of the variability in advanced AMD risk (Fritsche et al., Nat Genet. 2016; 48(2): 134-143). Many AMD-associated genetic alterations and variations reside in and around genes encoding components of the complement cascade, such as the Regulator of Complement Activation (RCA) locus on chromosome 1q31.3 which comprises the complement factor H (CFH) and complement factor H related 1-5 (CFHR1-5) genes (Schramm, E C et al., Mol Immunol 2014, 61:118-125; McHarg, S et al., Mol Immunol 2015, 67:43-50; herein incorporated by reference in their entirety).

The proteins encoded by the CFH/CFHR1-5 genes exert complement regulatory functions. The CFH gene encodes two proteins; FH, the main plasma regulator of complement activation, and a smaller splice variant called FH-like protein 1 (FHL-1) which predominates in the BrM and extracellular matrix of the choriocapillaris (Clark et al., J Immunol. 2014; 193(10):4962-70; McHarg et al., supra). The CFHR1-5 genes encode a group of five secreted plasma proteins (FHR-1 to FHR-5) synthesised primarily by hepatocytes. The FHRs retain some sequence homology with C3b binding domains of FH and are thought to enhance complement activation (Skerka et al., Mol Immunol. 2013, 56:170-180).

‘Dry’ AMD, also known as geographic atrophy, represents around 90% of late-stage AMD cases. In the remaining percentage of late-stage cases, the presence of drusen promotes choroidal neovascularisation (CNV), where the increased synthesis of vascular endothelial growth factor (VEGF) by RPE cells promotes new blood vessel growth from the choroid/choriocapillaris that breaks through BrM into the retina. These new blood vessels leak and eventually form scar tissue; this is referred to as ‘wet’ (neovascular or exudative) AMD. ‘Wet’ AMD, while only representing ˜10% of cases, is the most virulent form of late-stage AMD and has different disease characteristics to ‘dry’ AMD. There are treatments for wet AMD, where for example the injection of anti-VEGF agents into the vitreous of the eye can slow or reverse the growth of these blood vessels, although it cannot prevent their formation in the first place. Geographic atrophy (‘dry’ AMD) remains untreatable.

The present invention has been devised in light of the above considerations.

### SUMMARY OF THE INVENTION

The present invention provides methods for determining whether a subject is at risk of developing a complement-related disorder, using FHR-4 levels as a biomarker.

In one aspect, the present invention provides a method for determining whether a subject is at risk of developing a complement-related disorder, the method comprising determining the level of FHR-4 in the blood of said subject.

In some embodiments, the method comprises determining an increase in the level of FHR-4 in the blood of the subject. In some embodiments, an increased level of FHR-4 indicates an increased risk of developing a complement-related disorder.

In some embodiments, the method comprises determining the amount of FHR-4 in the blood of the subject. In various embodiments, the method comprises measuring the concentration of FHR-4 protein in the blood of said subject. In some embodiments, a FHR-4 concentration of >15 μg/ml indicates a high risk of said subject developing said disorder. In other embodiments, a FHR-4 concentration of 5-15 μg/ml indicates a medium risk of said subject developing the disorder, and/or an FHR-4 concentration of <5 μg/ml indicates a low risk of said subject developing the disorder.

In some embodiments, the level, amount and/or concentration of FHR-4 is determined in a blood-derived sample from the subject. In certain embodiments, the method comprises obtaining a blood-derived sample or biological sample from the subject.

In various embodiments, the level, amount and/or concentration of FHR-4 is determined in vitro.

In some embodiments, the methods comprise determining the level of expression of a gene encoding FHR-4 in said subject.

In another aspect, the present invention provides a method for determining whether a subject is at risk of developing a complement-related disorder, the method comprising determining the level of expression of a gene encoding FHR-4 in said subject. In some embodiments, an increased level of expression of a gene encoding FHR-4 indicates an increased risk of said subject developing the disorder. In some embodiments, an increased level of expression of a gene encoding FHR-4 when compared to a reference level of expression of a gene encoding FHR-4 indicates an increased risk of said subject developing the disorder.

In various embodiments, the complement-related disorder is selected from macular degeneration, age-related macular degeneration (AMD), geographic atrophy (dry′ (i.e. non-exudative) AMD), early AMD, intermediate AMD, late/advanced AMD, ‘wet’ (neovascular or exudative) AMD, choroidal neovascularisation (CNV), early-onset macular degeneration (EOMD), macular dystrophy, glaucoma, diabetic retinopathy, Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease. In some embodiments, the method further comprises determining in the subject the presence or absence of one or more genetic factors associated with AMD and/or EOMD.

In some embodiments, any of the methods provided herein may comprise a treatment step to treat or prevent the complement-related disorder. In some embodiments, the treatment step comprises administering to the subject a complement-targeted therapeutic and/or an agent that decreases the level of FHR-4 and/or decreases expression of a gene encoding FHR-4.

In another aspect, the present invention provides a complement-targeted therapeutic for use in a method of treating or preventing a complement-related disorder in a subject, wherein the subject has an increased level of FHR-4 and/or an increased level of a gene encoding FHR-4. Also provided is a method for treating or preventing a complement-related disorder in a subject, the method comprising administering an effective amount of a complement-targeted therapeutic to the subject, wherein the subject to be treated has an increased level of FHR-4 and/or an increased level of expression of a gene encoding FHR-4.

In some embodiments, the subject has been determined to have an increased level of FHR-4 and/or an increased level of expression of a gene encoding FHR-4.

In a further aspect, the present invention provides an agent that decreases the level of FHR-4 and/or decreases expression of a gene encoding FHR-4 for use in a method of treating or preventing a complement-related disorder in a subject, wherein the subject has an increased level of FHR-4 and/or an increased level of expression of a gene encoding FHR-4. Also provided is a method for treating or preventing a complement-related disorder in a subject, the method comprising administering an effective amount of an agent that decreases the level of FHR-4 and/or decreases expression of a gene encoding FHR-4 to the subject, wherein the subject has an increased level of FHR-4 and/or an increased level of expression of a gene encoding FHR-4.

In some embodiments, the subject has been determined to have an increased level of FHR-4 and/or an increased level of expression of a gene encoding FHR-4. In some embodiments, an increased level of FHR-4 indicates an increased risk of developing a complement-related disorder.

In some embodiments, the method comprises determining the amount of FHR-4 in the blood of the subject, optionally in vitro. In various embodiments, the method comprises measuring the concentration of FHR-4 protein in the blood of said subject, optionally in vitro. In some embodiments, a FHR-4 concentration of >15 μg/ml indicates a high risk of said subject developing said disorder. In other embodiments, a FHR-4 concentration of 5-15 μg/ml indicates a medium risk of said subject developing the disorder, and/or an FHR-4 concentration of <5 μg/ml indicates a low risk of said subject developing the disorder.

In various embodiments, an agent that decreases the level of FHR-4 and/or decreases expression of a gene encoding FHR-4 possesses one or more of the following properties: inhibits expression of the CFHR4 gene, degrades FHR-4 mRNA, binds to FHR-4 protein, sequesters FHR-4 protein, sequesters FHR-4 protein in the blood, competes for binding of FHR-4 protein, blocks activity of FHR-4 protein, reduces the concentration of FHR-4 in the blood, reduces the ability of FHR-4 protein to leave the blood, reduces the ability of FHR-4 protein to reach the eye, reduces the amount of FHR-4 in the eye, reduces the ability of FHR-4 protein to enter BrM, inhibits FHR-4-mediated signalling, modulates a reaction involving C3b, modulates a reaction involving FHR-4 and C3b, reduces the ability of FHR-4 protein to bind to C3b, competes with FHR-4 protein for C3b binding, encourages dissociation of FHR-4 from C3b, reduces C3 convertase activation, reduces production of C3bBb, increases C3 deactivation, increases production of iC3b, decreases complement activation, and/or inactivates a complement pathway.

In some embodiments, the agent is selected from: antisense nucleic acid, aptamer, antigen binding molecule, sequestering agent, and/or decoy receptor.

In various embodiments, the complement-related disorder is selected from macular degeneration, age-related macular degeneration (AMD), geographic atrophy (dry′ (i.e. non-exudative) AMD), early AMD, intermediate AMD, late/advanced AMD, ‘wet’ (neovascular or exudative) AMD, choroidal neovascularisation (CNV), early-onset macular degeneration (EOMD), macular dystrophy, glaucoma, diabetic retinopathy, Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease.

Also provided is a method of diagnosing the risk of onset of a complement-related disorder, the risk of disease progression of a complement-related disorder, and/or the presence of a complement-related disorder in a subject, the method comprising determining the level of FHR-4 and/or the level of expression of a gene encoding FHR-4 in the blood of the subject. In some embodiments, the method additionally comprises administering an effective amount of a complement-targeting therapeutic or an agent that decreases the amount of FHR-4 and/or decreases expression of a gene encoding FHR-4 if the subject has an increased level of FHR-4 and/or an increased level of a gene encoding FHR-4. In some embodiments, the method comprises obtaining a blood sample from the subject and measuring the level of FHR-4/the level of expression of a gene encoding FHR-4 in the sample.

In another aspect, the present invention provides a method for selecting a subject for treatment with a complement-targeted therapeutic or an agent that decreases the level of FHR-4 and/or decreases expression of a gene encoding FHR-4 to the subject, the method comprising determining the level of FHR-4 and/or the level of expression of a gene encoding FHR-4 in the subject and, optionally, where the level of FHR-4 and/or the level of expression of a gene encoding FHR-4 is increased, selecting the subject for treatment with the therapeutic or agent.

Also provided is a method for determining whether a subject having or suspected of having a complement-related disorder is likely to respond to treatment with a complement-targeted therapeutic or an agent that decreases the level of FHR-4 and/or decreases the level of expression of a gene encoding FHR-4, the method comprising determining the level of FHR-4 in the patient's blood and/or determining the level of expression of a gene encoding FHR-4 in the patient.

Also provided is a method for determining whether a subject is responding to therapeutic treatment with a complement-targeted therapeutic or an agent that decreases the level of FHR-4 and/or decreases the level of expression of a gene encoding FHR-4, the method comprising determining the level of FHR-4 in the blood of the subject after treatment is administered.

In another aspect, the present invention provides an agent that decreases the level of FHR-4 and/or decreases expression of a gene encoding FHR-4 for use in a method of treating or preventing an age-related macular degeneration (AMD) or early-onset macular degeneration (EOMD) in a subject. In some embodiments, the AMD is selected from geographic atrophy (‘dry’ (i.e. non-exudative) AMD), early AMD, intermediate AMD, late/advanced AMD, ‘wet’ (neovascular or exudative) AMD, and choroidal neovascularisation (CNV). In some embodiments, the agent that decreases the amount of FHR-4 and/or decreases expression of a gene encoding FHR-4 is a sequestering agent and/or decoy receptor for FHR-4.

The invention includes the combination of the aspects and preferred features described except where such a combination is clearly impermissible or expressly avoided.

### DETAILED DESCRIPTION OF THE INVENTION

The present invention concerns detecting, diagnosing and treating complement-related disorders. Provided herein are methods for determining the risk of onset or progression of disorders driven by complement activation, as well as methods for treating or preventing such disorders. In some aspects, provided are methods for determining the risk of development of macular degeneration, such as age-related macular degeneration (AMD) and early-onset macular degeneration (EOMD), as well as methods of treating or preventing AMD/EOMD.

The inventors have determined that FHR-4 is a positive regulator of complement activation. FHR-4 is shown herein to prevent FH-mediated C3b breakdown that will stimulate the formation of C3 convertase and the progression of the complement amplification loop. High levels of FHR-4 in tissues are likely to promote local inflammatory responses and cell lysis, leading to disorders associated with complement activation.

The inventors have determined that FHR-4 is not synthesised locally in tissues affected by complement activation, e.g. the eye in AMD. Instead, FHR-4 is expressed in the liver and is then transported around the body in the blood. The inventors have found that circulating FHR-4 levels can be used as an indicator of risk of developing complement-related disorders, even if the ultimate site of pathological complement activation is or will be located in a specific tissue. Treatment to reduce complement activation and/or to reduce the levels of FHR-4 may reduce the risk of development of, or ameliorate, complement-related diseases.

The development of many complement-related disorders is associated with a multitude of genetic alterations and/or variations in the patient. Genetic variants in and around many genes can be associated with a particular disorder. There can be a large amount of variation in the number and type of genetic alterations in patients suffering from the same disorder. For example, AMD risk has been associated with a variety of SNPs in over 34 genetic loci (Fritsche et al., Nat Genet. 2016; 48(2): 134-143). Thus, it may prove difficult to treat all patients with complement-related disorders in the traditional “one size fits all” manner.

Therefore, the present invention also concerns the determination of patient sub-groups that are expected to be responsive to treatment with a complement-targeted agent or an agent to reduce FHR-4 levels to treat complement-related disorders. The inventors have determined that high levels of systemic FHR-4 are indicative of increased AMD risk in a specific subset of AMD sufferers. The efficacy of treatments that target the complement system or the levels of FHR-4 can be maximised by first identifying those patients who display high levels of FHR-4.

C3b and FHR-4

The protein C3 plays a central role in the complement system and contributes to innate immunity. The three complement pathways, classical, alternative and lectin, all lead to the cleavage of C3 into C3a, a potent anaphylatoxin, and C3b. If C3b is not inactivated, it can associate with factor B and form the alternative pathway C3 convertase C3bBb. C3b activation of complement may occur on acellular structures, such as BrM and the intercapillary septa of the choriocapillaris. Active C3 convertase promotes a positive feedback cycle, referred to as the amplification loop. If left to continue unchecked, this cycle will stimulate the initiation of the terminal pathway of complement, which leads to inflammatory responses and cell lysis.

Cells have a number of cell surface proteins that are capable of down-regulating the complement cascade. C3b can be inactivated into iC3b by factor I (FI) and associated soluble cofactors such as factor H (FH) and factor H-like protein 1 (FHL-1). iC3b is unable to participate in C3 convertase assembly and is an opsonin, able to mediate leukocyte recruitment and debris removal. iC3b is broken down further into C3c and C3d, the latter of which plays a role in enhancing B cell responses.

The FHR proteins have been proposed to act as positive regulators of complement activation, allowing C3 convertase formation and driving the amplification loop.

Human complement factor H-related protein 4 (FHR-4 or CFHR-4; Uniprot: Q92496, Entry version 145 (28 Feb. 2018), Sequence version 3 (22 Jan. 2014)) belongs to the factor H family of plasma glycoproteins comprising short consensus repeat (SCR) domains (also known as sushi domains or complement control protein (CCP) domains).

FHR-4 is detected in human plasma as two different glycoproteins: a 578 amino acid (86-kDa) long isoform termed FHR-4A (Uniprot: Q92496-1; SEQ ID NO:1) composed of 9 SCRs and a 331 amino acid (˜45-kDa) shorter isoform FHR-4B (Uniprot Q92496-3; SEQ ID NO:3) composed of 5 SCRs corresponding to SCRs 1 and 6-9 of FHR-4A. A second isoform of FHR-4A (SEQ ID NO:2) has a deletion (Glu) at position 20. Human FHR-4 contains a 19 amino acid signal peptide which is cleaved to yield the mature FHR-4 protein (SEQ ID NO:5; SEQ ID NO:6) The FHR-4 proteins are encoded by the CFHR4 gene (NCBI Gene ID: 10877).

As used herein, the term “FHR-4” includes at least one of FHR-4A isoform 1, FHR-4A isoform 2 or FHR-4B, and preferably includes FHR-4A isoforms 1 and 2 as well as FHR-4B. “FHR-4” refers to FHR-4 from any species and includes isoforms, fragments, variants or homologues of FHR-4 from any species. In preferred embodiments, “FHR-4” refers to human FHR-4.

The FHR-4 isoforms lack SCRs homologous to the N-terminal complement inhibitory domains SCR1-4 of FH and FHR-1. However, the FHR-4 proteins do share homology with the C-terminal FH domains SCR19-20 that contain C3b/C3d-binding sites and both FHR-4A and FHR-4B have been shown to bind to C3b (Hellwage J., FEBS Lett. 1999, 462, 345-352 and Hellwage J., J. Immunol. 2002, 169, 6935-6944; Hebecker and Józsi, J Biol Chem. 2012, 287(23):19528-36). Furthermore, FHR-4 reportedly serves as a platform for the assembly of C3 convertase (Hebecker and Józsi, J Biol Chem. 2012, 287(23):19528-36).

Methods for Assessing Complement-Related Disorders

In some aspects, the present invention provides methods for assessing the risk of onset, or risk of progression, of a complement-related disorder using systemic FHR-4 levels.

The methods may be diagnostic, prognostic and/or predictive of the risk of onset or progression of a complement-related disorder. Diagnostic methods can be used to determine the diagnosis or severity of a disease, prognostic methods help to predict the likely course of disease in a defined clinical population under standard treatment conditions, and predictive methods predict the likely response to a treatment in terms of efficacy and/or safety, thus supporting clinical decision-making.

Methods of the present invention use the level of systemic FHR-4 as a biomarker to determine whether a subject is at risk of developing a complement-related disorder. The terms “disorder”, “disease” and “condition” may be used interchangeably and refer to a pathological issue of a body part, organ or system which may be characterised by an identifiable group of signs or symptoms. The term “complement-related disorder” refers to disorders, diseases or conditions that comprise or arise from deficiencies or abnormalities in the complement system. In some embodiments, the complement-related disorder is a disorder driven by complement activation or complement over-activation.

The complement-related disorder may comprise disruption of the classical, alternative and/or lectin complement pathways. In some cases, the disorder may be associated with deficiencies in regulatory components of the complement system. In some embodiments, the disorder may be a disorder associated with the alternative complement pathway, disruption of the alternative complement pathway and/or associated with deficiencies in regulatory components of the alternative complement pathway. In some cases, the disorder is associated with one or more of C3, C3b, FH, FHL-1, FI, CR1, CD46, CD55, C4BP, Factor B (FB), Factor D (FD), FHR-1, FHR-2, FHR-3, FHR-5, SPICE, VCP (or VICE) and/or MOPICE. In some cases, the disorder is associated with deficiencies or abnormalities in the activity of one or more of C3, C3b, FH, FHL-1, FI, CR1, CD46, CD55, C4BP, Factor B, Factor D, FHR-1, FHR-2, FHR-3, FHR-5, SPICE, VCP (or VICE) and/or MOPICE, or where one or more of these proteins are pathologically implicated.

In some embodiments, the disorder may be a disorder associated with C3 or a C3-containing complex, an activity/response associated with C3 or a C3-containing complex, or a product of an activity/response associated with C3 or a C3-containing complex. That is, in some embodiments, the disorder is a disorder in which C3, a C3-containing complex, an activity/response associated with C3 or a C3-containing complex, or the product of said activity/response is pathologically implicated. In some embodiments, the disorder may be associated with an increased level of C3 or a C3-containing complex, an increased level of an activity/response associated with C3 or a C3-containing complex, or increased level of a product of an activity/response associated with C3 or a C3-containing complex as compared to the control state.

In some embodiments, the disorder may be a disorder associated with C3b or a C3b-containing complex, an activity/response associated with C3b or a C3b-containing complex, or a product of an activity/response associated with C3b or a C3b-containing complex. That is, in some embodiments, the disorder is a disorder in which C3b, a C3b-containing complex, an activity/response associated with C3b or a C3b-containing complex, or the product of said activity/response is pathologically implicated. In some embodiments, the disorder may be associated with an increased level of C3b or a C3b-containing complex, an increased level of an activity/response associated with C3b or a C3b-containing complex, or increased level of a product of an activity/response associated with C3b or a C3b-containing complex as compared to the control state.

In some embodiments, the disorder may be a disorder associated with FH, FHL-1, FI, FB, FD, CR1 and/or CD46, an activity/response associated with FH, FHL-1, FI, FB, FD, CR1 and/or CD46 or a product of an activity/response associated with FH, FHL-1, FI, FB, FD, CR1 and/or CD46. In some embodiments, the disorder is a disorder in which FH, FHL-1, FI, FB, FD, CR1 and/or CD46, an activity/response associated with FH, FHL-1, FI, FB, FD, CR1 and/or CD46, or the product of said activity/response is pathologically implicated. In some embodiments, the disorder may be associated with a decreased level of FH, FHL-1, FI, FB, FD, CR1 and/or CD46, a decreased level of an activity/response associated with FH, FHL-1, FI, FB, FD, CR1 and/or CD46, or a decreased level of a product of an activity/response associated with FH, FHL-1, FI, FB, FD, CR1 and/or CD46 as compared to a control state.

In some embodiments the disorder is associated with increased levels of C3, C3b, C3 convertase and/or C3bBb as compared to a control state. In some embodiments, the disorder is associated with decreased levels of iC3b as compared to a control state.

The disorder may be an ocular disorder. In some embodiments, the disease or condition to be treated or prevented is a complement-related ocular disease. In some embodiments, the disease or condition to be treated or prevented is macular degeneration. In some embodiments, the disorder may be selected from, i.e. is one or more of, age-related macular degeneration (AMD), choroidal neovascularisation (CNV), macular dystrophy, and diabetic maculopathy. As used herein, the term “AMD” includes early AMD, intermediate AMD, late/advanced AMD, geographic atrophy (‘dry’ (i.e. non-exudative) AMD), and ‘wet’ (i.e. exudative or neovascular) AMD, each of which may be a disorder in its own right that is detected, treated and/or prevented as described herein. In some embodiments the disease or condition to be treated or prevented is a combination of the diseases/conditions above, e.g. ‘dry’ and ‘wet’ AMD. In some embodiments the disease or condition to be treated or prevented is not ‘wet’ AMD or choroidal neovascularisation. AMD is commonly-defined as causing vision loss in subjects age 50 and older. In some embodiments a subject to be treated is age 50 or older, i.e. is at least 50 years old.

As used herein “early AMD” refers to a stage of AMD characterised by the presence of medium-sized drusen, commonly having a diameter of up to ˜200 μm, within Bruch's membrane adjacent to the RPE layer. Subjects with early AMD typically do not present with significant vision loss. As used herein “intermediate AMD” refers to a stage of AMD characterised by large drusen and/or pigment changes in the retina. Intermediate AMD may be accompanied by some vision loss. As used herein “late AMD” refers to a stage of AMD characterised by the presence of drusen and vision loss, e.g. severe central vision loss, due to damage to the macula. In all stages of AMD, ‘reticular pseudodrusen’ (RPD) or ‘reticular drusen’ (also referred to as subretinal drusenoid deposits (SDD)) may be present, referring to the accumulation of extracellular material in the subretinal space between the neurosensory retina and RPE. “Late AMD” encompasses ‘dry’ and ‘wet’ AMD. In ‘dry’ AMD (also known as geographic atrophy), there is a gradual breakdown of the light-sensitive cells in the macula that convey visual information to the brain and of the supporting tissue beneath the macula. In ‘wet’ AMD (also known as choroidal neovascularization, neovascular and exudative AMD), abnormal blood vessels grow underneath and into the retina. These vessels can leak fluid and blood which can lead to swelling and damage of the macula and subsequent scar formation. The damage may be rapid and severe.

In some embodiments the disease or condition to be treated or prevented is early-onset macular degeneration (EOMD). As used herein “EOMD” refers to a phenotypically severe sub-type of macular degeneration that demonstrates a much earlier age of onset than classical AMD and results in many more years of substantial visual loss. Sufferers may show an early-onset drusen phenotype comprising uniform small, slightly raised, yellow subretinal nodules randomly scattered in the macular, also known as ‘basal laminar drusen’ or ‘cuticular drusen’. EOMD may also be referred to as “middle-onset macular degeneration”. The EOMD subset is described in e.g. Boon C J et al. Am J Hum Genet 2008; 82(2):516-23, van de Ven J P, et al. Arch Ophthalmol 2012; 130(8):1038-47, and Taylor R L et al, Ophthalmology. 2019 Mar. 21. pii: S0161-6420(18):33171-3, each of which is hereby incorporated by reference in its entirety. As with other types of macular degeneration, EOMD is related to complement dysregulation and disrupted Factor H activity. In some embodiments a subject to be treated is age 49 or younger. In some embodiments a subject to be treated is between ages 15 and 49, i.e. is between 15 and 49 years old. In some embodiments the disease or condition to be treated is a macular dystrophy. A macular dystrophy is a genetic condition, usually caused by a mutation in a single gene, that results in degeneration of the macula.

In some embodiments the disorder may be selected from Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease.

In some cases, the disorder is a neurological and/or neurodegenerative disorder.

In one aspect, the present invention provides a method for determining whether a subject is at risk of developing a complement-related disorder, the method comprising determining the level of FHR-4 in the blood of said subject. In some cases, the method comprises determining an increase in the level of FHR-4 in the blood of said subject. In some cases, an increase in the level of FHR-4 indicates an increased risk of developing the disorder. The method may be used for determining whether a subject is at risk of onset of the disorder, and/or is at risk of progression, exacerbation or worsening of the disorder.

The “level of FHR-4” may be the level, amount, or concentration of FHR-4. In some embodiments, the methods provided herein determine the level of circulating or systemic FHR-4. The term “biomarker(s)” as used herein refers to one or more measurable indicators of a biological state or condition. The terms “develop”, “developing”, and “development”, e.g. of a disorder, as used herein refer both to the onset of a disease as well as the progression, exacerbation or worsening of a disease state.

In another aspect, the present invention provides a method for determining whether a subject is at risk of developing macular degeneration, e.g. EOMD and/or AMD, the method comprising determining the level of FHR-4 in the blood of said subject. In some cases, the method comprises determining an increase in the level of FHR-4 in the blood of said subject. In some cases, an increase in the level of FHR-4 indicates an increased risk of developing the disorder.

The method may be used for determining whether a subject is at risk of onset of macular degeneration, e.g. EOMD and/or AMD, and/or is at risk of EOMD and/or AMD progression. In some cases, the disorder is selected from EOMD, AMD, geographic atrophy (dry′ (i.e. non-exudative) AMD), early AMD, intermediate AMD, late/advanced AMD, ‘wet’ (neovascular or exudative) AMD, choroidal neovascularisation (CNV) and retinal dystrophy.

In other aspects, the present invention provides a method for identifying a subject at risk of developing a complement-related disorder, the method comprising determining the level of FHR-4 in the blood of said subject. The disorder may be EOMD and/or AMD.

In some embodiments, the methods provided herein comprise determining an increase in the level of FHR-4 in the blood of a subject, wherein the level of FHR-4 is compared to a reference value. In some embodiments, an increase in the level of FHR-4 in the blood of a subject compared to a reference value indicates an increased risk of a subject developing a complement-related disorder.

In some embodiments, the methods provided herein comprise determining an increase in the amount of FHR-4 in the blood of a subject. For example, a method may comprise measuring the amount of FHR-4 in the blood of said subject. In some embodiments, an increased amount of FHR-4 in the blood indicates an increased risk of said subject developing the disorder. In some cases, an increased amount of FHR-4 in the blood when compared to a reference value indicates an increased risk of said subject developing the disorder.

In some embodiments, an increased amount of FHR-4 is an FHR-4 concentration of 5-10 μg/ml, 10-15 μg/ml, 15-20 μg/ml or >20 μg/ml. That is, an FHR-4 concentration of 5-10 μg/ml, 10-15 μg/ml, 15-20 μg/ml or >20 μg/ml indicates an increased risk of said subject developing the disorder. In preferred embodiments, an increased amount of FHR-4 is >15 μg/ml. In other preferred embodiments, an increased amount of FHR-4 is >20 μg/ml. In some embodiments, an FHR-4 concentration of >15 μg/ml indicates a high risk of said subject developing the disorder, an FHR-4 concentration of 5-15 μg/ml indicates a medium risk of said subject developing the disorder, and/or an FHR-4 concentration of <5 μg/ml indicates a low risk of said subject developing the disorder.

In some cases, an FHR-4 concentration of 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml or greater, indicates an increased risk of a subject developing the disorder.

The present invention also provides methods that employ the level of expression of a gene encoding FHR-4 as a biomarker to determine whether a subject is at risk of developing a complement-related disorder. Thus, in some aspects, provided is a method for determining whether a subject is at risk of developing a complement-related disorder, the method comprising determining the level of expression of a gene encoding FHR-4 in said subject. In some embodiments, an increased level of expression of a gene encoding FHR-4 indicates an increased risk of said subject developing the disorder. In some embodiments, an increased level of expression of a gene encoding FHR-4 when compared to a reference level of expression of a gene encoding FHR-4 indicates an increased risk of said subject developing the disorder. In some cases, the method comprises measuring the level of expression of a gene encoding FHR-4. In some embodiments, the gene encoding FHR-4 is CFHR4. In some embodiments, the method comprises determining/measuring the level of expression of a gene encoding FHR-4 and determining the level of FHR-4, as described above.

Any method provided herein may comprise determining the level or amount of FHR-4, and/or the level of expression of a gene encoding FHR-4, in a sample from a subject. A sample may be taken from any tissue or bodily fluid. In preferred arrangements the sample is taken from a bodily fluid, more preferably one that circulates through the body. Accordingly, the sample may be a blood sample or lymph sample. In a particularly preferred arrangement the sample is a blood sample or blood-derived sample. The blood-derived sample may be a selected fraction of a patient's blood, e.g. a selected cell-containing fraction or a plasma or serum fraction. A selected serum fraction may comprise the fluid portion of the blood obtained after removal of the fibrin clot and blood cells. Alternatively the sample may comprise or may be derived from a tissue sample, biopsy or isolated cells from said individual.

In some embodiments, the sample comprises tissue from the liver, and/or liver cells. In some embodiments, the sample comprises circulating immune cells e.g. isolated immune cells. In some cases, the sample may comprise one or more monocytes, macrophages, neutrophils, eosinophils, basophils, mast cells, and/or lymphocytes e.g. NK cells, T cells and/or B cells.

Thus, in some aspects, the present invention provides a method for determining whether a subject is at risk of developing a complement-related disorder, the method comprising determining the level or amount of FHR-4, and/or the level of expression of a gene encoding FHR-4, in a sample from said subject. The sample may be the same sample, or may comprise more than one sample e.g. one from blood, one from a tissue.

In some embodiments, any method provided herein comprises a first stage of obtaining a blood-derived sample or biological sample from the subject.

In some cases, the methods provided herein comprise determining the level or amount of FHR-4 and determining the level of expression of a gene encoding FHR-4. The two determining steps may be performed on the same sample or in different samples from a subject. In some cases, any method provided herein may comprise quantifying the amount of FHR-4 and/or the level of expression of a gene encoding FHR-4.

Methods according to the present invention may be performed outside the human or animal body. Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism. In some embodiments, the determining, detecting, measuring, quantifying and/or diagnosing steps of the methods provided herein are performed in vitro.

In some embodiments, the present invention provides a method for determining whether a subject is at risk of developing a complement-related disorder comprising one or more of the steps of: (i) obtaining a blood-derived sample from the subject; (ii) contacting the sample with an antibody which binds specifically to FHR-4; and (iii) detecting the amount of FHR-4 present in the sample. In some embodiments, the method comprises detecting binding between FHR-4 and the antibody. In some embodiments, the method comprises (i) providing a blood-derived sample from a subject, (ii) contacting the sample with an antibody which binds specifically to FHR-4; (iii) detecting the amount of FHR-4 present in the sample. The method may comprise detecting, measuring or quantifying the concentration of FHR-4, as described hereinabove.

The antibody may be any suitable FHR-4 antibody known in the art or commercially available, for example: MAB5980, IC5980G, AF5980 from R & D Systems; MA5-24288, PA5-41991 from Invitrogen; or PA5-41991 from ThermoFisher Scientific. FHR-4 antibodies may also be produced by techniques as described herein, or known in the art, see e.g. Chiu and Gilliland, Curr Opin Struct Biol. 2016, 38:163-173, Jakobovits A Curr Opin Biotechnol. 1995 October;6(5):561-6, and Brüggemann M et al., Arch Immunol Ther Exp (Warsz). 2015; 63(2): 101-108. One suitable technique is phage display technology, see e.g. Hammers and Stanley, J Invest Dermatol. 2014, 134(2): e17 and Bazan J et al., Hum Vaccin Immunother. 2012, 8(12): 1817-1828. Antigen-binding polypeptide chains may also be produced by techniques such as chemical synthesis (see e.g. Chandrudu et al., Molecules (2013), 18: 4373-4388), recombinant expression such as the techniques set out in Green and Sambrook, Molecular Cloning: A Laboratory Manual (4th Edition), Cold Spring Harbor Press, 2012, and in Nat Methods. (2008); 5(2): 135-146, or cell-free-protein synthesis (CFPS; see e.g., Zemella et al. Chembiochem (2015) 16(17): 2420-2431), all of which are hereby incorporated by reference in their entirety.

Methods for detecting, measuring or quantifying the amount of FHR-4 using an antibody are known in the art, and include e.g. ELISA, see e.g. Crowther J R, Methods in Molecular Biology, The ELISA Guidebook. Second Edition. Humana Press, a part of Springer Science+Business Media, LLC 2009; Butler J. E. The Behaviour of Antigens and Antibodies Immobilized on a Solid Phase. In: M. H. V. Van Regenmortel, ed. Structure of Antigens. Boca Raton, Fla.: CRC Press, 1992: 209-259. Vol. 1, 209; CRC Press, Inc.; Lequin R M. Clinical chemistry 51.12 (2005): 2415-2418; and Engvall and Perlmann. Immunochemistry 8.9 (1971): 871-874, which are hereby incorporated by reference in their entirety. Other methods may include mass spectrometry, Western blotting, protein immunostaining, immunoelectrophoresis, and protein immunoprecipitation, which are described hereinbelow and/or will be apparent to one skilled in the art.

Also provided herein is a method for assessing the propensity or predisposition of a subject to develop a complement-related disorder, comprising:
(a) providing a blood sample from the subject;
(b) assessing the level of FHR-4 in the sample;
(c) using the results of (b) to determine the likelihood of the subject to develop a complement-related disorder.

In some cases, an increased level of FHR-4 indicates an increased risk of said subject developing the disorder. In some cases, an increased amount of FHR-4 in the blood when compared to a reference value indicates an increased risk of said subject developing the disorder. Concentrations of FHR-4 and associated grades of risk are as described hereinabove. Methods for assessing the level of FHR-4 in the sample are as described hereinbelow.

As used herein the term “reference value” refers to a known measurement value used for comparison during analysis. In some cases, the reference value is one or a set of test values obtained from an individual or group in a defined state of health. In some cases, the reference value is/has been obtained from determining the level of FHR-4 in subjects known not to have a complement-related disorder. In some cases, the reference value is set by determining the level or amount of FHR-4 previously from the same subject e.g. at an earlier stage of disease progression. The reference value may be a standard value, standard curve or standard data set.

The methods provided herein may comprise determining in a subject the presence or absence of a genetic profile characterised by polymorphisms in the subject's genome associated with complement dysregulation. The polymorphisms may be found within or near genes such as CCL28, FBN2, ADAM12, PTPRC, IGLC1, HS3ST4, PRELP, PPID, SPOCK, APOB, SLC2A2, COL4A1, MYOC, ADAM19, FGFR2, C8A, FCN1, IFNAR2, C1NH, C7 and ITGA4. A genetic profile associated with complement dysregulation may comprise one or more, often multiple, single nucleotide polymorphisms, e.g. as set out in Tables I and II of US 2010/0303832, which is herein incorporated by reference in its entirety.

Genetic factors are thought to play a role in the development of AMD and EOMD. Thus, any of the assessment or therapeutic methods described herein may be performed in conjunction with methods to assess AMD-associated and/or EOMD-associated and/or macular dystrophy-associated genetic variants.

In some cases, the methods provided herein further comprise determining in a subject the presence or absence of one or more genetic factors associated with AMD, e.g. one or more AMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors.

In some embodiments, the one or more genetic factors may be located on chromosome 1 at the CFH/CFHR locus.

The one or more genetic factors may be located in one or more of: CFH e.g. selected from Y402H (i.e. rs1061170c), rs1410996c, 162V (r5800292), A473A (r52274700), R53C, D90G, D936E (rs1065489), R1210C, IVS1 (rs529825), IVS2 insTT, IVS6 (rs3766404), A307A (rs1061147), IVS10 (rs203674), rs3753396, R1210C, rs148553336, rs191281603, rs35292876, and rs800292; CFHR4 e.g. selected from rs6685931, and rs1409153; CFI e.g. selected from G119R, and rs141853578; CFB e.g. rs4151667, C2 e.g. rs9332739, C9 e.g. P167S; and/or C3 e.g. K155Q. In some embodiments, a genetic factor is Y402H (i.e. rs1061170c). In some embodiments, a genetic factor is rs3753396. In some embodiments, a genetic factor is rs6685931 and/or rs1409153. In some embodiments, a genetic factor is not rs6685931.

In some cases, the genetic factor is located in the CFHR4 gene.

Suitable genetic risk factors and genetic variants will be known in the art and may be as described in e.g. Edwards A O et al., Science 2005, 308(5720):421-4; Hageman G S et al., Proc Natl Acad Sci USA. 2005, 102(20):7227-7232; Haines J L et al., Science 2005, 308(5720):419-21, Klein R J et al., Science 2005, 308(5720):385-389; Fritsche et al., Nat Genet. 2016, 48(2):134-43; US 2010/0303832; or Clark et al., J Clin Med. 2015, 4(1):18-31, each herein incorporated by reference in its entirety.

In some cases, the methods provided herein further comprise determining in a subject the presence or absence of one or more genetic factors associated with EOMD, e.g. one or more EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors. In some embodiments the subject may possess one or more risk factors for early-onset macular degeneration (EOMD).

EOMD is thought to be caused by monogenic inheritance of rare variants of the CFH gene (see e.g. Boon C J et al. Am J Hum Genet 2008; 82(2):516-23; van de Ven J P, et al. Arch Ophthalmol 2012; 130(8):1038-47; Yu Y et al. Hum Mol Genet 2014; 23(19):5283-93; Duvvari M R, et al. Mol Vis 2015; 21:285-92; Hughes A E, et al. Acta Ophthalmol 2016; 94(3):e247-8; Wagner et al. Sci Rep 2016; 6:31531; Taylor R L et al, Ophthalmology. 2019 Mar. 21. pii: 50161-6420(18):33171-3). In some embodiments, the subject may possess one or more of EOMD-associated genetic variants. EOMD-associated genetic variants are described in e.g. Servais A et al. Kidney Int, 2012; 82(4):454-64 and Dragon-Durey M A, et al. J Am Soc Nephrol 2004; 15(3):787-95; which are hereby incorporated by reference in their entirety. In some embodiments, the subject may possess one or more of the following EOMD-associated genetic variants: CFH c.1243de1, p.(Ala415Profs*39) het; CFH c.350+1G>T het; CFH c.619+1G>A het; CFH c.380G>A, p.(Arg127His); CFH c.694C>T, p.(Arg232Ter); or CFH c.1291T>A, p.(Cys431Ser).

In some cases, the methods comprise screening for deletions within the RCA locus (a region of DNA sequence located on chromosome one that extends from the CFH gene through to the CD46 (MCP) gene) that are associated with AMD risk or protection.

In some cases, provided herein is a method comprising determining the presence or absence of genetic factors associated with increased FHR-4 levels and/or increased expression of a gene encoding FHR-4. In some cases, a method comprises determining the presence or absence of genetic factors associated with a risk of increased FHR-4 levels and/or a risk of increased expression of a gene encoding FHR-4.

In some cases, where a subject is/has been determined to have increased levels of FHR-4 and/or increased expression of a gene encoding FHR-4, a method provided herein comprises determining the presence or absence of genetic factors associated with said increase(s). That is, the methods of the present invention may comprise determining the presence or absence of genetic factors associated with an increase in FHR-4 level and/or an increase in the level of expression of a gene encoding FHR-4, in order to confirm that said level(s) are a consequence of or are associated with genetic variation.

Methods for determining the presence or absence of genetic factors include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, PCR, DNA sequencing e.g. Sanger sequencing or Next-Generation sequencing, allele specific oligonucleotide (ASO) probes, and oligonucleotide microarrays or beads. Other suitable methods are described in e.g. Edenberg H J and Liu Y, Cold Spring Harb Protoc; 2009; doi:10.1101/pdb.top62, and Tsuchihashi Z and Dracopoli N C, Pharmacogenomics J., 2002, 2:103-110.

Methods provided herein for assessing the risk of development, i.e. the onset or risk of progression, of a complement-related disorder may be performed in conjunction with additional diagnostic methods and/or tests for such disorders that will be known to one skilled in the art. In some cases, methods for assessing the risk of development of a complement-related disorder comprise further techniques selected from CH50 or AH50 measurement via haemolytic assay, measurement of neoantigen formation during MAC complex (C789) generation, C3 deficiency screening, mannose-binding lectin assays, immunochemical assays to quantify individual complement components, flow cytometry to assess cell-bound regulatory proteins e.g. CD55, CD59 and CD35, renal function tests and determining plasma levels and/or levels of complement regulatory proteins and/or complement activation (e.g. levels of C3, C4, CFH, CFI), see e.g. Shih A R and Murali M R, Am. J. Hematol. 2015, 90(12):1180-1186, Ogedegbe H O, Laboratory Medicine, 2007, 38(5):295-304, and Gowda S et al., N Am J Med Sci. 2010, 2(4): 170-173, which are herein incorporated by reference in their entirety.

In some cases, methods provided herein for assessing the risk of development of AMD and/or EOMD comprise further assessment techniques selected from dark adaptation testing, contrast sensitivity testing e.g. Pelli Robson, visual acuity testing using e.g. a Snellen chart and/or Amsler grid, Farnsworth-Munsell 100 hue test and Maximum Color Contrast Sensitivity test (MCCS) for assessing colour acuity and colour contrast sensitivity, preferential hyperacuity perimetry (PHP), fundus photography of the back of the eye, fundus examination, fundus autofluorescence, optical coherence tomography, angiography e.g. fluorescence angiography, fundus fluorescein angiography, indocyanine green angiography, optical coherence tomography angiography, electroretinogram methods, and/or methods to measure histological changes such as atrophy, retinal pigment changes, exudative changes e.g. hemorrhages in the eye, hard exudates, subretinal/sub-RPE/intraretinal fluid, and/or the presence of drusen.

Therapeutic and Prophylactic Applications

Methods according to the present invention provide means for identifying a specific subset of patients that have an increased level of FHR-4 and who therefore may be at risk of developing a complement-related disorder. Selected patients may be treated accordingly for a complement-related disorder. Methods described herein may also be useful for selecting subjects for therapeutic or prophylactic treatment, determining whether a subject is responding to a therapeutic treatment, and/or determining whether a subject is likely to respond or not respond to a therapeutic or prophylactic treatment e.g. a treatment for a complement-related disorder.

Thus in some aspects, a subject who is at risk of developing a complement-related disorder, who has a complement-related disorder, who has been determined to be at risk of and/or have a complement-related disorder, who has increased FHR-4 levels and/or increased expression of a gene encoding FHR-4, or who has been determined to have increased FHR-4 levels and/or increased expression of a gene encoding FHR-4, e.g. by a method as described herein, may benefit from treatment for a complement-related disorder.

Any method provided herein for determining whether a subject is at risk of developing a complement-related disorder may additionally comprise a treatment step to treat said disorder. For example, a method provided herein for determining whether a subject is at risk of developing a complement-related disorder may comprise a treatment step to treat or prevent said disorder, wherein the subject has been determined to have increased FHR-4 levels and/or increased expression of a gene encoding FHR-4.

In one aspect, provided herein is a method for determining whether a subject is at risk of developing a complement-related disorder and treating said disorder, the method comprising determining the level of FHR-4 and/or the level of expression of a gene encoding FHR-4 in the blood of said subject, and administering to the subject an effective amount of a complement-targeted therapeutic wherein the subject has an increased level of FHR-4 and/or an increased level of a gene encoding FHR-4. Methods of measuring FHR-4 and grades of risk are as described herein.

A treatment step may comprise administering to a subject a therapeutically or prophylactically effective amount of one or more complement-targeted therapeutics, for example, one or more C1 inhibitors, C5 inhibitors, C5a inhibitors, CSaR antagonists, C3 inhibitors, C3a inhibitors, C3b inhibitors, C3aR antagonists, classical pathway inhibitors, alternative pathway inhibitors, FH-supplementation therapy and/or MBL pathway inhibitors. Specific complement-targeted therapeutics include without limitation one or more of human C1 esterase inhibitor (C1-INH), eculizumab (Soliris®, Alexion; a humanized monoclonal IgG2/4-antibody targeting C5), APL-2 (Apellis), mubodina (Adienne Pharma and Biotech), ergidina (Adienne Pharma and Biotech), rituximab (Biogen Idec, Genentech, Hoffmann-La Roche), ofatumumab (Genmab, GSK), compstatin analogues, soluble and targeted forms of CD59, PMX53 and PMX205, (Cephalon/Teva), JPE-1375 (Jerini), CCX168 (ChemoCentryx), NGD-2000-1 (former Neurogen), Cinryze (Shire), Berinert (CSL Behring), Cetor (Sanquin), Ruconest/Conestat alfa (Pharming), TNT009 (True North), OMS721 (Omeros), CLG561 (Novartis), AMY-101 (Amyndas), APL-1 (Apellis), APL-2 (Apellis), Mirococept (MRC), Lampalizumab (Genentech), ACH-4471 (Achillion), ALXN1210 (Alexion), Tesidolumab/LFG316 (Novartis/Morphosys), Coversin (Akari), RA101495 (Ra Pharma), Zimura (Ophthotech), ALN-CC5 (Alnylam), IFX-1 (InflaRx), ALXN1007 (Alexion), Avacopan/CCX168 (Chemocentryx) and/or one or more therapeutic agents as described in e.g. Ricklin et al., Mol Immunol. 2017, 89:10-21; Ricklin and Lambris, Adv Exp Med Biol. 2013, 734: 1-22; Ricklin and Lambris, Semin Immunol. 2016, 28(3):208-22; Melis J P M et al., Mol Immunol. 2015 67(2):117-130; Thurman J M, Nephrol Dial Transplant, 2017 32: i57-i64, Cashman S M et al., PLoS One. 2011, 6(4):e19078; Bora N S et al., J Biol Chem. 2010, 285(44):33826-33, which are herein incorporated by reference in their entirety.

In some aspects, the present invention provides a method for treating or preventing a complement-related disorder in a subject, the method comprising administering an effective amount of a complement-targeted therapeutic, wherein the subject to be treated has an increased level of FHR-4 and/or an increased level of a gene encoding FHR-4.

In other aspects, the present invention provides a complement-targeted therapeutic for use in a method of treating or preventing a complement-related disorder in a subject, wherein the subject has an increased level of FHR-4 and/or an increased level of a gene encoding FHR-4. In further aspects, provided is the use of a complement-targeted therapeutic in the manufacture of a medicament for treating or preventing a complement-related disorder in a subject, wherein the subject has an increased level of FHR-4 and/or an increased level of a gene encoding FHR-4.

Also provided is a method of treating or preventing a complement-related disorder in a subject, or a complement-targeted therapeutic for use in a method of treating or preventing a complement-related disorder in a subject, the method comprising administering an effective amount of a complement-targeted therapeutic wherein the subject is selected for treatment if the subject has an increased level of FHR-4 and/or an increased level of a gene encoding FHR-4.

The present invention also provides a method for selecting a patient for treatment with a complement-targeted therapeutic, comprising determining the level of FHR-4 and/or the level of expression of a gene encoding FHR-4 in the blood of said subject. The patient may have, or have been determined to have, a complement-related disorder, e.g. by methods provided herein.

In various aspects provided herein, the subject to be treated has an increased level of FHR-4 and/or an increased level of expression of a gene encoding FHR-4. In some embodiments, the subject to be treated has been determined to have an increased level of FHR-4 and/or has been determined to have an increased level of expression of a gene encoding FHR-4. As described hereinabove, the level of FHR-4 or expression of a gene encoding FHR-4 may be, or have been, determined using any method provided herein. In some embodiments, the subject has a high, medium or low risk of developing a complement-related disorder, as described herein. Said methods may include determining e.g. in a sample the amount or concentration of FHR-4, determining the presence or absence of a genetic profile associated with complement dysregulation, and/or further methods for assessing the risk of developing a complement-related disorder, as described herein. In various aspects, a subject may have, or have been determined to have, an increased level of FHR-4. In other embodiments a subject may have, or have been determined to have, an increased level of expression of a gene encoding FHR-4. In some embodiments, a subject may have, or have been determined to have, an increased level of FHR-4 and an increased level of expression of a gene encoding FHR-4. In some embodiments, the gene is CFHR4.

According to some aspects of the present invention, a subject who is at risk of developing a complement-related disorder, who has a complement-related disorder, who has been determined to be at risk of and/or have a complement-related disorder, who has increased FHR-4 levels and/or increased expression of a gene encoding FHR-4, or who has been determined to have increased FHR-4 levels and/or increased expression of a gene encoding FHR-4, e.g. by a method as described herein, may benefit from treatment to reduce the level of FHR-4 and/or reduce the level of expression of a gene encoding FHR-4.

Thus any method provided herein for determining whether a subject is at risk of developing a complement-related disorder may additionally comprise a treatment step comprising administering to the subject an effective amount of an agent that decreases the level of FHR-4 and/or decreases the level of expression of a gene encoding FHR-4.

In some aspects, the present invention provides a method for determining whether a subject is at risk of developing a complement-related disorder and treating said disorder, the method comprising determining the level of FHR-4 and/or the level of expression of a gene encoding FHR-4 in the blood of said subject, and administering to the subject an effective amount of a complement-targeted therapeutic wherein the subject has an increased level of FHR-4 and/or an increased level of a gene encoding FHR-4. Methods of measuring FHR-4 and grades of risk are described herein.

The present invention provides a method of treating or preventing a complement-related disorder in a subject, comprising administering to a subject a therapeutically or prophylactically effective amount of an agent that decreases the level of FHR-4 and/or decreases the level expression of a gene encoding FHR-4. Also provided is an agent that decreases the level of FHR-4 and/or decreases the level of expression of a gene encoding FHR-4 for use in a method of treating or preventing a complement-related disorder in a subject. Also provided is the use of an agent that decreases the level of FHR-4 and/or decreases the level of expression of a gene encoding FHR-4 in the manufacture of a medicament for treating or preventing a complement-related disorder. In some cases, an agent is administered to a subject in need thereof.

In various embodiments the subject has, or has been determined to have e.g. by methods provided herein, a complement-related disorder and/or an increased risk of developing a complement-related disorder. In some embodiments, the subject has a high, medium or low risk of developing a complement-related disorder, as described herein. In some embodiments, the subject has, or has been determined to have e.g. by methods provided herein, an increased level of FHR-4 and/or an increased level of expression of a gene encoding FHR-4.

The present invention provides methods for identifying subjects for treatment for a complement-related disorder. Thus, provided herein is a method of selecting a subject for treatment or prevention of a complement-related disorder, the treatment comprising administering an effective amount of an agent that decreases the level of FHR-4 and/or decreases the level of expression of a gene encoding FHR-4, wherein the subject is selected for treatment if the subject has an increased level of FHR-4 and/or an increased level of a gene encoding FHR-4. The subject may have, or have been determined to have e.g. by methods provided herein, a complement-related disorder and/or an increased level of FHR-4 and/or an increased level of expression of a gene encoding FHR-4.

Also provided is an agent that decreases the level of FHR-4 and/or decreases the level of expression of a gene encoding FHR-4 for use in a method of treating or preventing a complement-related disorder in a subject, wherein the subject is selected for treatment if the subject has an increased level of FHR-4 and/or an increased level of a gene encoding FHR-4. The subject may have, or have been determined to have e.g. by methods provided herein, a complement-related disorder, an increased level of FHR-4 and/or an increased level of expression of a gene encoding FHR-4. The method may comprise administering an effective amount of the agent to the subject.

The present invention also provides a method for selecting a patient for treatment with an agent that decreases the amount of FHR-4 and/or decreases expression of a gene encoding FHR-4, comprising determining the level of FHR-4 and/or the level of expression of a gene encoding FHR-4 in the blood of said subject. The patient may have, or have been determined to have, a complement-related disorder, e.g. by methods provided herein. The patient may be selected for treatment according to any method provided herein.

In various aspects provided herein, the subject to be treated has an increased level of FHR-4 and/or an increased level of expression of a gene encoding FHR-4. In some embodiments, the subject to be treated has been determined to have an increased level of FHR-4 and/or has been determined to have an increased level of expression of a gene encoding FHR-4. As described hereinabove, the level of FHR-4 or expression of a gene encoding FHR-4 may be, or have been, determined using any method provided herein. Said methods may include determining e.g. in a sample, the amount or concentration of FHR-4, determining of the presence or absence of a genetic profile associated with complement dysregulation, and/or further methods for assessing the risk of developing a complement-related disorder, as described herein. In some embodiments, the subject has a high, medium or low risk of developing a complement-related disorder, as described herein. In various aspects, a subject may have, or have been determined to have, an increased level of FHR-4. In other embodiments a subject may have, or have been determined to have, an increased level of expression of a gene encoding FHR-4. In some embodiments, a subject may have, or have been determined to have, an increased level of FHR-4 and an increased level of expression of a gene encoding FHR-4. In some embodiments, the gene is CFHR4.

Agents suitable for decreasing the amount of FHR-4 and/or decreasing the level of expression of a gene encoding FHR-4 are described hereinbelow and/or are known in the art.

Complement-related disorders which may be treated or prevented according to the present invention are described hereinabove, including disorders associated with one or more of C3b, FH, FHL-1, FI, CR1, CD46, CD55, C4BP, Factor B (FB), Factor D (FD), FHR-1, FHR-2, FHR-3, FHR-5, SPICE, VCP (or VICE) and/or MOPICE.

In some cases, the methods described herein find use in treating or preventing, or selecting a patient for treatment or prevention of, a disorder which would benefit from one or more of: a reduction in the level or activity of C3bBb-type C3 convertase, C3bBb3b-type C5 convertase or C4b2a3b-type C5 convertase; a reduction in the level of C3b, C5b or CSa; or an increase in the level of iC3b, C3f, C3dg or C3d.

The disorder to be treated or prevented may be selected from macular degeneration, early-onset macular degeneration (EOMD), age-related macular degeneration (AMD), geographic atrophy (dry′ (i.e. non-exudative) AMD), early AMD, intermediate AMD, late/advanced AMD, ‘wet’ (neovascular or exudative) AMD, choroidal neovascularisation (CNV), macular dystrophy, glaucoma, diabetic retinopathy, diabetic maculopathy, Haemolytic Uremic Syndrome (HUS), atypical Haemolytic Uremic Syndrome (aHUS), autoimmune uveitis, Membranoproliferative

Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease.

In some embodiments, the disorder to be treated or prevented is selected from macular degeneration, early-onset macular degeneration (EOMD), age-related macular degeneration (AMD), geographic atrophy (dry′ (i.e. non-exudative) AMD), early AMD, intermediate AMD, late/advanced AMD, ‘wet’ (neovascular or exudative) AMD, choroidal neovascularisation (CNV) and macular dystrophy.

As used herein, ‘treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention/prophylaxis of a disease/condition may refer to prevention of a worsening of the condition or prevention of the development of the disease/condition, e.g. preventing an early stage disease/condition developing to a later, chronic, stage.

Also provided is a method of diagnosing the risk of onset of a complement-related disorder, the risk of disease progression of a complement-related disorder, and/or the presence of a complement-related disorder in a subject, the method comprising determining the level of FHR-4 and/or the level of expression of a gene encoding FHR-4 in the blood of the subject. The method additionally comprises administering an effective amount of a complement-targeting therapeutic or an agent that decreases the amount of FHR-4 and/or decreases expression of a gene encoding FHR-4 if the subject has an increased level of FHR-4 and/or an increased level of a gene encoding FHR-4. In some embodiments, the method comprises obtaining a blood sample from the subject and measuring the level of FHR-4/the level of expression of a gene encoding FHR-4 in the sample. In any aspect of the present invention provided herein, the method may comprise a step of correlating the presence of an increased level of FHR-4 and/or an increased level of expression of a gene encoding FHR-4 with an increased risk of the subject developing a complement-related disorder and/or having a complement-related disorder. Suitable methods and techniques for determining the level of FHR-4 are described herein.

In some embodiments, the subject is selected for therapeutic or prophylactic treatment with an agent that decreases the level of FHR-4 and/or decreases the level of expression of a gene encoding FHR-4 based on their being determined to possess one or more genetic factors for AMD and/or EOMD, e.g. one or more AMD-associated and/or EOMD-associated genetic variants or a macular dystrophy. In some embodiments, the subject has been determined to have one or more such genetic factors. In some embodiments, the method comprises determining whether a subject possesses one or more such genetic factors. Such methods and genetic factors are described herein. Thus, provided herein is a method of treating or preventing a complement-related disorder in a subject, the method comprising administering an agent that decreases the level of FHR-4 and/or decreases the level of expression of a gene encoding FHR-4, wherein the subject has been determined to possess one or more genetic factors for AMD and/or EOMD.

In some aspects, the present invention provides methods employing FHR-4 levels for determining whether a subject is likely to respond or not respond to a therapeutic treatment, or whether a subject is responding to a therapeutic treatment. Such methods should enable patients to receive the most effective therapy for their particular pathological requirements.

Thus, there is provided a method for determining whether a subject having or suspected of having a complement-related disorder is likely to respond to treatment with a complement-targeted therapeutic or an agent that decreases the level of FHR-4 and/or decreases the level of expression of a gene encoding FHR-4, the method comprising determining the level of FHR-4 in the patient's blood and/or determining the level of expression of a gene encoding FHR-4 in the patient. In some cases, if the level of FHR-4 and/or the level of expression of a gene encoding FHR-4 is increased, then the patient is likely to respond to treatment with complement-targeted therapeutic or an agent that decreases the level of FHR-4 and/or decreases the level of expression of a gene encoding FHR-4. In some cases, where the level of FHR-4 and/or the level of expression of a gene encoding FHR-4 is increased, the subject is selected for treatment with the therapeutic or agent. Methods for determining the level of FHR-4 or the level of expression of a gene encoding FHR-4 may be as described herein.

Also provided is a method for selecting a subject for treatment with a complement-targeted therapeutic or an agent that decreases the level of FHR-4 and/or decreases the level of expression of a gene encoding FHR-4, the method comprising determining the level of FHR-4 in the blood of the subject and/or determining the level of expression of a gene encoding FHR-4, and, optionally, where the level of FHR-4 and/or the level of expression of a gene encoding FHR-4 is increased, selecting the subject for treatment with the therapeutic or agent.

In some cases, the subject has or is suspected to have a complement-related disorder. In some cases the disorder is AMD. In some cases the disorder is EOMD.

Also provided is a method for determining whether a subject is responding to therapeutic treatment with a complement-targeted therapeutic or an agent that decreases the level of FHR-4 and/or decreases the level of expression of a gene encoding FHR-4, the method comprising determining the level of FHR-4 in the blood of the subject after treatment is administered. In some cases, the method comprises additionally determining the level of FHR-4 in the blood of the subject before treatment, wherein a decrease in the level of FHR-4 after treatment as compared to the level of FHR-4 before treatment indicates that the subject is responding/has responded to said treatment. In some cases, a decrease in the level of FHR-4 after treatment as compared to a reference value indicates that the subject is responding/has responded to said treatment

In any of the methods described herein, the level of FHR-4 may be determined in a biological sample and/or measured as described herein. Any method provided herein may comprise determining the level of FHR-4 in vitro. An increased amount of FHR-4 may be a FHR-4 concentration of 5-10 μg/ml, 10-15 μg/ml, 15-20 μg/ml or >20 μg/ml. In some embodiments, a method provided herein comprises a step of correlating the presence of an increased amount of FHR-4 with an increased risk of the subject developing AMD and/or EOMD. Any method provided herein may comprise quantifying the amount of FHR-4 and/or the level of expression of CFHR4.

The term “subject” refers to a subject, patient or individual and may be any animal or human. The subject is preferably mammalian, more preferably human. The subject may be a non-human mammal, but is more preferably human. The subject may be male or female. The subject may be a patient. Therapeutic uses may be in human or animals (veterinary use). The subject to be treated with a therapeutic substance described herein may be a subject in need thereof.

A subject described herein may belong to a patient subpopulation i.e. the subject may be part of an identifiable, specific portion or subdivision of a population. The population and/or subpopulation may have or be suspected to have a complement-related disorder. The subpopulation may display increased levels of FHR-4 and/or increased levels of expression of a gene encoding FHR-4 as compared to the population as a whole. The population and/or subpopulation may have or be suspected to have AMD, EOMD or a macular dystrophy.

In some aspects, provided is a method of treating or preventing a complement-related disorder in a subject, wherein the subject is characterised in having an increased level of FHR-4 and/or an increased level of expression of a gene encoding FHR-4.

Also provided is a complement-targeted therapeutic or an agent that decreases the level of FHR-4 and/or decreases the level of expression of a gene encoding FHR-4 for use in a method of treating or preventing a complement-related disease in a subject, wherein the subject is characterised in having an increased level of FHR-4 and/or an increased level of expression of a gene encoding FHR-4.

Agents Targeting FHR-4/CFHR4

Subjects with increased levels of FHR-4 and/or expression of a gene encoding FHR-4 may derive therapeutic or prophylactic benefit from said levels being reduced. This may be achieved by administering any suitable agent that decreases the level of FHR-4 and/or decreases the level of expression of a gene encoding FHR-4.

Thus, in some embodiments the methods provided herein comprise administering an agent that decreases the level of FHR-4 and/or decreases the level of expression of a gene encoding FHR-4. In some embodiments, the agent is capable of decreasing the level of FHR-4 and/or capable of decreasing the level of expression of a gene encoding FHR-4.

In some embodiments, the agent decreases the level of FHR-4. In other embodiments, the agent decreases the level of expression of a gene encoding FHR-4. In some cases, the agent decreases the level of FHR-4 and the level of expression of a gene encoding FHR-4. In other cases, the methods comprise administering a first agent that decreases the level of FHR-4 and a second agent that decreases the level of expression of a gene encoding FHR-4. Alternatively, the methods may comprise administering a first agent that decreases the level of expression of a gene encoding FHR-4 and a second agent that decreases the level of FHR-4. The term “level of FHR-4” includes for example the amount or concentration of FHR-4. The level of FHR-4 and/or the level of expression of a gene encoding FHR-4 may be decreased in a subject, e.g. in the blood of a subject and/or in the liver of a subject.

The agent may possess one or more of the following properties: acts to inhibit expression of the CFHR4 gene, degrades FHR-4 mRNA, binds to FHR-4 protein, sequesters FHR-4 protein, sequesters FHR-4 protein in the blood, competes for binding of FHR-4 protein, blocks activity of FHR-4 protein, reduces the concentration of FHR-4 in the blood, reduces the ability of FHR-4 protein to leave the blood, reduces the ability of FHR-4 protein to reach the eye, reduces the amount of FHR-4 in the eye, reduces the ability of FHR-4 protein to enter BrM, inhibits FHR-4-mediated signalling, modulates a reaction involving C3b, modulates a reaction involving FHR-4 and C3b, reduces the ability of FHR-4 protein to bind to C3b, competes with FHR-4 protein for C3b binding, encourages dissociation of FHR-4 from C3b, reduces C3 convertase activation, reduces production of C3bBb, increases C3 deactivation, increases production of iC3b, decreases complement activation, and/or inactivates a complement pathway e.g. alternative complement pathway.

Herein, “inhibits”, “inhibition”, “reduces” or “reduction” refers to a reduction, decrease or lessening relative to a control condition. Herein, “decreases the level of FHR-4” refers to reduction or lessening relative to a control condition. The level of FHR-4 may be measured by determining the level, amount or concentration of FHR-4 in the blood of a subject relative to a reference level. A decrease in the level of FHR-4 and/or a decrease in the level of expression of a gene encoding FHR-4 may be measured by determining the level of FHR-4/the level of expression of a gene encoding FHR-4 in the subject after treatment with the agent and comparing it to the level in the subject before treatment. The decrease in the level of FHR-4 may also refer to the sequestration or binding of FHR-4 by agents in such a way that circulating levels/amount/concentration of FHR-4 are decreased. Thus, in some cases, an agent may be described as an agent that decreases the level of circulating FHR-4.

In some embodiments, the agent that decreases the amount of FHR-4 or decreases expression of a gene encoding FHR-4 is an antisense nucleic acid. An “antisense nucleic acid” as referred to herein is a nucleic acid (e.g. DNA or RNA molecule) that is complementary to at least a portion of a specific target nucleic acid (e.g. an mRNA translatable into a protein) and is capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Antisense nucleic acids are capable of hybridizing to (e.g. selectively hybridizing to) a target nucleic acid (e.g. target mRNA). In some cases, the antisense nucleic acid hybridizes to the target nucleic acid sequence (e.g. mRNA) under stringent hybridization conditions. In some cases, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and anomeric sugar-phosphate backbone modified nucleotides.

In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (see e.g. Marcus-Sakura, Anal. Biochem. 1988, 172:289). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors. Antisense nucleic acid molecules may stimulate RNA interference (RNAi).

Thus, an antisense nucleic acid may interfere with transcription of CFHR4, interfere with translation of FHR-4 mRNA and/or promote degradation of FHR-4 mRNA. In some cases, an antisense nucleic acid is capable of inducing a reduction in expression of the CFHR4 gene.

An antisense nucleic acid may target any region of a gene encoding FHR-4. In some cases, the gene is CFHR4. In some cases, an antisense nucleic acid may target one or more of the nucleotide sequences in SEQ ID NO:10, 12, 14, 16, 18 or 20. In some cases, an antisense nucleic acid targets the nucleotide sequences in SEQ ID NO:12 and/or 14. These antisense nucleic acids may be described as siRNA molecules.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

RNAi and siRNA are described in, for example, Dana et al., Int J Biomed Sci. 2017; 13(2): 48-57, herein incorporated by reference in its entirety. An antisense nucleic acid molecule may contain double-stranded RNA (dsRNA) or partially double-stranded RNA that is complementary to a target nucleic acid sequence, for example FHR-4. A double-stranded RNA molecule is formed by the complementary pairing between a first RNA portion and a second RNA portion within the molecule. The length of an RNA sequence (i.e. one portion) is generally less than 30 nucleotides in length (e.g. 29, 28, 27, 26, 25, 24, 23, 22, 21, 20, 19, 18, 17, 16, 15, 14, 13, 12, 11, 10 or fewer nucleotides). In some embodiments, the length of an RNA sequence is 18 to 24 nucleotides in length. In some siRNA molecules, the complementary first and second portions of the RNA molecule form the “stem” of a hairpin structure. The two portions can be joined by a linking sequence, which may form the “loop” in the hairpin structure. The linking sequence may vary in length and may be, for example, 5, 6, 7, 8, 9, 10, 11, 12, or 13 nucleotides in length. Suitable linking sequences are known in the art.

Suitable siRNA molecules for use in the methods of the present invention may be designed by schemes known in the art, see for example Elbashire et al., Nature, 2001 411:494-8; Amarzguioui et al., Biochem. Biophys. Res. Commun. 2004 316(4):1050-8; and Reynolds et al., Nat. Biotech. 2004, 22(3):326-30. Details for making siRNA molecules can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine internet website). Typically, a number of siRNAs are generated and screened to obtain an effective drug candidate, see, U.S. Pat. No. 7,078,196. siRNAs can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

The nucleic acid molecule may be a miRNA. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length)

The nucleic acid molecule may be an aptamer. The term “aptamer” as used herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington A D, Szostak J W, Nature 1990, 346:818-822; Tuerk C, Gold L. Science 1990, 249:505-510) or by developing SOMAmers (slow off-rate modified aptamers) (Gold L et al. (2010) Aptamer-based multiplexed proteomic technology for biomarker discovery. PLoS ONE 5(12):e15004). An aptamer suitable for use as described herein may display specific binding for FHR-4. The aptamer may inhibit the function of FHR-4, for example blocking FHR-4 binding to C3b.

In some embodiments, the agent that decreases the amount of FHR-4 or decreases expression of a gene encoding FHR-4 is an antibody or antigen-binding molecule (both referred to herein as “antigen-binding molecule”) e.g. an anti-FHR-4 antibody. In some cases, the antigen-binding molecule is specific for FHR-4. In some cases, the antigen-binding molecule displays specific binding to FHR-4. In some cases, the antigen-binding molecule is specific for C3b. In some cases, the antigen-binding molecule displays specific binding to C3b. As used herein, “specific binding” refers to binding which is selective for the antigen, and which can be discriminated from non-specific binding to non-target antigen. An antigen-binding molecule that specifically binds to a target molecule preferably binds the target with greater affinity, and/or with greater duration than it binds to other, non-target molecules. In some cases, the antigen-binding molecule displays specific binding for FHR-4 over FHR-1, FHR-2, FHR-3 and/or FHR-5, or over FH and/or FHL-1. An antigen-binding molecule may bind to human FHR-4 with a KD of 1 μM or less, preferably one of 1 μM, 100 nM, 10 nM, 1 nM or 100 μM.

Anti-FHR-4 antigen-binding molecules may be antagonist antigen-binding molecules that inhibit or reduce a biological activity of FHR-4. Anti-FHR-4 antigen-binding molecules may be neutralising antigen-binding molecules that neutralise the biological effect of FHR-4, e.g. its ability to stimulate production of C3 convertase via C3b.

The antigen-binding molecule may bind to a particular region of interest of FHR-4 or C3b. The antigen-binding region of an antigen-binding molecule may bind to a linear epitope of FHR-4 or C3b, consisting of a contiguous sequence of amino acids (i.e. an amino acid primary sequence). In some embodiments, the antigen-binding region molecule may bind to a conformational epitope of FHR-4 or C3b, consisting of a discontinuous sequence of amino acids of the amino acid sequence.

The antigen-binding molecule may be a multispecific antigen-binding molecule. By “multispecific” it is meant that the antigen-binding molecule displays specific binding to more than one target. In some embodiments the antigen-binding molecule is a bispecific antigen-binding molecule. In some embodiments the antigen-binding molecule comprises at least two different antigen-binding domains (i.e. at least two antigen-binding domains, e.g. comprising non-identical VHs and VLs). Multispecific antigen-binding molecules may be provided in any suitable format, such as those formats described in described in Brinkmann and Kontermann MAbs (2017) 9(2): 182-212, which is hereby incorporated by reference in its entirety.

In some embodiments the antigen-binding molecule binds to FHR-4 and another target (e.g. an antigen other than FHR-4), and so is at least bispecific. The term “bispecific” means that the antigen-binding molecule is able to bind specifically to at least two distinct antigenic determinants.

The ability of a given polypeptide to bind specifically to a given molecule or another given peptide/polypeptide can be determined by analysis according to methods known in the art, such as by ELISA, Surface Plasmon Resonance (SPR; see e.g. Hearty et al., Methods Mol Biol 2012, 907:411-442), Bio-Layer Interferometry (see e.g. Lad et al., J Biomol Screen. 2015, 20(4): 498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay. Binding affinity may be expressed in terms of dissociation constant (KD).

The region of a peptide/polypeptide to which an antibody binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21(3):145-156, which is hereby incorporated by reference in its entirety.

In some embodiments, the antigen-binding molecule decreases the concentration of FHR-4 in the blood. In some embodiments, the antigen-binding molecule decreases the amount of circulating FHR-4, e.g. in the blood. In some embodiments, the antigen-binding molecule may sequester FHR-4 protein. In some embodiments, the antigen-binding molecule binds to FHR-4 and reduces the ability of FHR-4 to reach the eye, enter the BrM, and/or enter the intercapillary septa of the choriocapillaris. In some embodiments, the antigen-binding molecule reduces binding of FHR-4 to C3b.

The ability of an antigen-binding molecule to inhibit interaction between two binding partners can also be determined by analysis of the downstream functional consequences of such interaction. For example, the ability of an antigen-binding molecule to inhibit interaction of FHR-4 and C3b may be determined by analysis of production of C3bBb and/or iC3b in an appropriate assay e.g. by detecting the production of protein from a reaction using ELISA, Western blotting or by electrophoresis methods such as those described herein.

A person skilled in the art will be able to produce suitable antigen binding molecules using e.g. techniques as described herein or those known in the art, see e.g. Chiu and Gilliland, Curr Opin Struct Biol. 2016, 38:163-173, Jakobovits A, Curr Opin Biotechnol. 1995 October;6(5):561-6, and Brüggemann Metal., Arch Immunol Ther Exp (Warsz). 2015; 63(2): 101-108. One suitable technique is phage display technology, see e.g. Hammers and Stanley, J Invest Dermatol. 2014, 134(2): e17 and Bazan J et al., Hum Vaccin Immunother. 2012, 8(12): 1817-1828. Antigen-binding polypeptide chains may also be produced by techniques such as chemical synthesis (see e.g. Chandrudu et al., Molecules (2013), 18: 4373-4388), recombinant expression such as the techniques set out in Green and Sambrook, Molecular Cloning: A Laboratory Manual (4th Edition), Cold Spring Harbor Press, 2012, and in Nat Methods. (2008); 5(2): 135-146, or cell-free-protein synthesis (CFPS; see e.g., Zemella et al. Chembiochem (2015) 16(17): 2420-2431), all of which are hereby incorporated by reference in their entirety. The antigen-binding molecule may be monoclonal.

Examples of known anti-FHR-4 antibodies/antigen-binding molecules are described hereinabove.

An agent that decreases the amount of FHR-4 and/or decreases expression of a gene encoding FHR-4 may be a sequestering agent, e.g. of FHR-4. The agent may be a protein molecule. An example is an antigen-binding molecule e.g. as described herein, which sequesters FHR-4 in the blood.

A sequestering agent or antigen-binding molecule may bind to FHR-4 in the region of SCRs 4/8/9 for FHR-4A or SCRs 4/5 for FHR-4B (see e.g. Hebecker and JOzsi, J Biol Chem. 2012, 287(23):19528-36). For example, the agent or antigen-binding molecule may bind to a sequence between positions 456 and 578 of SEQ ID NO:1, positions 455 and 577 of SEQ ID NO:2, and/or positions 209 and 331 of SEQ ID NO:3.

The agent may be a small molecule. For example, the small molecule may bind to FHR-4 protein and prevent/reduce the ability of FHR-4 to reach sites of complement activation and/or prevent/reduce an interaction between FHR-4 and a normal binding partner e.g. C3b. The small molecule may prevent/reduce correct folding of the FHR-4 protein. In some cases, the small molecule prevents/reduces binding between FHR-4 and a binding partner. In some cases, the small molecule binds to FHR-4.

An agent that decreases the amount of FHR-4 and/or decreases expression of a gene encoding FHR-4 may be a decoy receptor. In some embodiments, a decoy receptor refers to a peptide or polypeptide capable of binding FHR-4. The receptor may be a receptor, including fragments and derivatives thereof, for FHR-4. A decoy receptor may be able to recognise and bind a specific ligand but may not be able to signal or activate a subsequent response. A decoy receptor may bind FHR-4 to form a complex. A decoy receptor may act as an inhibitor of FHR-4 by binding FHR-4 and preventing/reducing the ability or availability of FHR-4 to bind to a FHR-4 receptor. Thus the agent may be a molecule which binds FHR-4 so that FHR-4 is not available to activate C3b. The agent may be based on C3b, for example the receptor may be an inactive form of C3b. The agent may be based on SEQ ID NO:8 and/or SEQ ID NO:9. The agent may be based on C3c and/or C3d.

The agent may be administered to/present in the blood, or attached to a tissue e.g. in or near the eye. The receptor may be capable of inhibiting complement activation. The receptor may be capable of inhibiting interaction between FHR-4 and C3b. The receptor may be capable of inhibiting the activation of C3b, and/or inhibiting the formation of C3 convertase.

In some cases, the receptor is capable of inhibiting FHR-4 activity. In some cases, the decoy receptor may be a molecule comprising a region corresponding to the FHR-4 binding domain of C3b/C3d (see e.g. Hellwage J., FEBS Lett. 1999, 462, 345-352; Hellwage J., J. Immunol. 2002, 169, 6935-6944; Hebecker and Józsi, J Biol Chem. 2012, 287(23):19528-36; and Nagar B et al., Science 1998: 1277-1281, which are hereby incorporated by reference in their entirety).

A decoy receptor may be soluble (not membrane bound), or may be membrane bound e.g. expressed on a cell surface. Decoy receptors may be presented and/or administered on a surface of a nanocarrier, for example, a nanoparticle, liposome, bead, polymer, metal particle, dendrimer, nanotube or micro-sized silica rods, see e.g. Wilczewska A Z et al., Pharmacol Rep. 2012, 64(5):1020-1037.

Methods for detecting whether a decoy receptor competes for FHR-4 binding are described herein e.g. SPR (see e.g. Hearty et al., Methods Mol Biol 2012, 907:411-442), competition ELISA assay or solid phase binding assays. Other suitable methods will be known in the art.

Agents that decrease the amount of FHR-4 and/or decrease expression of a gene encoding FHR-4 may fall into more than one of the categories above. For example, an antigen binding molecule or decoy receptor may also be a sequestering agent.

Any of the agents described herein may be optionally isolated and/or substantially purified.

Administration of an agent that decreases the level of FHR-4 and/or decreases the level of expression of a gene encoding FHR-4, e.g. as described herein, is preferably in a “therapeutically effective amount” or a “prophylactically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the condition to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins. The agent may be administered in a therapeutically effective amount to a subject in need thereof.

Agents that decrease the level of FHR-4 and/or decrease the level of expression of a gene encoding FHR-4 may be formulated as pharmaceutical compositions or medicaments for clinical use and may comprise a pharmaceutically acceptable carrier, diluent, excipient or adjuvant. In accordance with the present invention methods are also provided for the production of pharmaceutically useful compositions, such methods of production may comprise one or more steps selected from: isolating an agent; and/or mixing an agent with a pharmaceutically acceptable carrier, adjuvant, excipient or diluent. The composition may be formulated for topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral or transdermal routes of administration which may include injection or infusion, or administration as an eye drop (i.e. ophthalmic administration). Suitable formulations may comprise the agent in a sterile or isotonic medium. Medicaments and pharmaceutical compositions may be formulated in fluid, including gel, form. Fluid formulations may be formulated for administration by injection or infusion (e.g. via catheter) to a selected organ or region of the human or animal body. A further aspect of the present invention relates to a method of formulating or producing a medicament or pharmaceutical composition for use in a method of medical treatment, the method comprising formulating a pharmaceutical composition or medicament by mixing an agent that decreases the level of FHR-4 and/or decreases the level of expression of a gene encoding FHR-4 as described herein with a pharmaceutically acceptable carrier, adjuvant, excipient or diluent.

In some cases, the agent is administered to the liver, e.g. to one or more hepatocytes. In some cases, the agent is administered to the blood (i.e. intravenous/intra-arterial administration). In some cases, the agent is administered subcutaneously.

In some cases, the methods comprise targeted delivery of the agent i.e. wherein the concentration of the agent in the subject is increased in some parts of the body relative to other parts and/or wherein the agent is delivered via a controlled-release technique. In some cases, the methods comprise intravenous, intra-arterial, intramuscular or subcutaneous administration and wherein the agent is formulated in a targeted agent delivery system. Suitable targeted agent delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and agent delivery systems will be apparent to one skilled in the art. In some cases, the agent is formulated for targeted delivery to a specific organ(s) or tissue(s). In some cases, the agent is delivered to the liver. In some cases, the methods comprise intravenous, intra-arterial, intramuscular or subcutaneous administration and wherein the agent is formulated for targeted delivery to the liver.

In some cases, RNA, e.g. nanoparticle based formulations, may be formulated for pulmonary administration for subsequent delivery to non-lung tissues, see e.g. US 2015/0157565 A1, which is herein incorporated in its entirety.

The particular mode and/or site of administration may be selected in accordance with the location where reduction of FHR-4 levels and/or reduction of the level of CFHR4 expression is required. In some cases, the methods comprise intravenous and/or intra-arterial administration. In some cases, the methods comprise administration to the eye. Should reduction of CFHR4 expression be required, then an agent that decreases expression of a gene encoding FHR-4 may be administered to the liver. In some cases, the agent is delivered to one or more hepatocytes.

Methods for RNA delivery are described herein and are known in the art and can be found, for example, in Tatiparti K et al. “siRNA Delivery Strategies: A Comprehensive Review of Recent Developments.” Ed. Thomas Nann. Nanomaterials 7.4 (2017): 77, and Lehto T et al., Adv Drug Deliv Rev. 2016, 106(Pt A):172-182, which are herein incorporated by reference in their entirety. For example, RNA may be delivered naked, or by using nanoparticles, polymers, peptides e.g. cell-penetrating peptides, or by ex vivo transfection. Nanoparticles may be organic, e.g. micelles, liposomes, proteins, solid-lipid particles, solid polymer particles, dendrimers, and polymer therapeutics. Nanoparticles may be inorganic such as nanotubes or metal particles, optionally with organic molecules added. Viruses present another nanoparticle delivery option. Nanoparticles may be optimised to improve rate of endocytosis, avoid renal clearance and filtration, improve thermal stability, improve pH stability, prevent toxic effects, and improve RNA loading efficiency. Further encapsulation methods are described in e.g. US 2015/0157675 A1.

Administration may be alone or in combination with other treatments (e.g. other therapeutic or prophylactic intervention), either simultaneously or sequentially dependent upon the condition to be treated. An agent that decreases the level of FHR-4 and/or decreases the level of expression of a gene encoding FHR-4 and another therapeutic agent may be administered simultaneously or sequentially. In some cases, an agent that decreases the level of FHR-4 and/or decreases the level of expression of a gene encoding FHR-4 is administered simultaneously or sequentially with a complement-targeted therapeutic e.g. as described herein.

Other therapeutic agents or techniques suitable for use with the present invention may comprise nutritional therapy, photodynamic therapy (PDT), laser photocoagulation, anti-VEGF (vascular endothelial growth factor) therapy, and/or additional therapies known in the art, see e.g. Al-Zamil W M and Yassin S A, Clin Interv Aging. 2017 Aug. 22; 12:1313-1330). Anti-VEGF therapy may comprise agents such as ranibizumab (Lucentis, made by Genentech/Novartis), Avastin (Genentech), bevacizumab (off label Avastin), and aflibercept (Eylea®/VEGF Trap-Eye from Regeneron/Bayer). Further agents or techniques suitable for use with the present invention include APL-2 (Apellis), AdPEDF (GenVec), encapsulated cell technology (ECT; Neurotech), squalamine lactate (EVIZON™, Genaera), OT-551 (antioxidant eye drops, Othera), anecortave actate (Retaane®, Alcon), bevasiranib (siRNA, Acuity Pharmaceuticals), pegaptanib sodium (Macugen®), and AAVCAGsCD59 (Clinical trial identifier: NCT03144999).

Simultaneous administration refers to administration of the agent that decreases the level of FHR-4 and/or decreases the level of expression of a gene encoding FHR-4 and another therapeutic agent together, for example as a pharmaceutical composition containing both agents (combined preparation), or immediately after each other and optionally via the same route of administration, e.g. to the same tissue, artery, vein or other blood vessel. Sequential administration refers to administration of one of the polypeptide, nucleic acid, vector, cell or composition or therapeutic agent followed after a given time interval by separate administration of the other agent. It is not required that the two agents are administered by the same route, although this is the case in some embodiments. The time interval may be any time interval.

Multiple doses of an agent that decreases the level of FHR-4 and/or decreases the level of expression of a gene encoding FHR-4 may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

An agent described herein may be formulated in a sustained release delivery system, in order to release the polypeptide, nucleic acid, vector or composition at a predetermined rate. Sustained release delivery systems may maintain a constant drug/therapeutic concentration for a specified period of time. In some embodiments, an agent described herein is formulated in a liposome, gel, implant, device, or drug-polymer conjugate e.g. hydrogel.

Determining Levels of Protein/Gene Expression

The amount of FHR-4, e.g. in a sample, may be measured using techniques well known in the art or as described herein.

For example, any method provided herein may employ an FHR-4 specific ELISA (enzyme-linked immunosorbent assay) to measure the amount of FHR-4. The amount of FHR-4 may be measured by determining the concentration of FHR-4. Methods for performing ELISA are well known in the art, see e.g. Crowther J R, Methods in Molecular Biology, The ELISA Guidebook. Second Edition. Humana Press, a part of Springer Science+Business Media, LLC 2009; Butler J. E. The Behaviour of Antigens and Antibodies Immobilized on a Solid Phase. In: M. H. V. Van Regenmortel, ed. Structure of Antigens. Boca Raton, Fla.: CRC Press, 1992: 209-259. Vol. 1, 209; CRC Press, Inc.; Lequin R M. Clinical chemistry 51.12 (2005): 2415-2418; and Engvall and Perlmann. Immunochemistry 8.9 (1971): 871-874, hereby incorporated by reference in their entirety. In some cases, the amount of FHR-4 is measured using a sandwich ELISA, e.g. as described herein.

Other suitable methods for detecting protein include: measuring the absorbance of a protein-containing sample, Bradford protein assay (see e.g. Bradford M, Anal Biochem. 1976, 72: 248-254), Biuret test derived assays e.g. Bicinchoninic acid assay (BCA assay; see e.g. Smith P K et al., Anal. Biochem. 1985, 150:76-85) or Lowry Protein assay (see e.g. Lowry O H et al., J. Biol. Chem. 1951, 193:265-275; Sargent M. Anal. Biochem. 1987, 163:476-481), fluorescamine techniques (see e.g. Böhlen P et al., Arch. Biochem. Biophys. 1973, 155:213-220), amido black techniques, colloidal gold techniques (see e.g. Zeng S et al., Plasmonics. 2011, 6(3):491-506; Tauran Y et al., World J Biol Chem. 2013, 4(3):35-63), high performance liquid chromatography (HPLC; see e.g. Thammana M, RRJPA 2016, 5(2):22-28; liquid chromatography-mass spectrometry (LC/MS; see e.g. Pitt J J, Clin Biochem Rev. 2009; 30(1):19-34), protein immunoprecipitation (see e.g. Burgess R R, Methods Enzymol. 2009; 463:331-42), immunoelectrophoresis (see e.g. Levinson, S. S. (2009). Immunoelectrophoresis. In eLS, (Ed.)), Western blot (see e.g. Mahmood and Yang, N Am J Med Sci. 2012; 4(9):429-434), protein immunostaining (see e.g. Ramos-Vara J A, Veterinary Pathology, 2005, 42(4):405-426), and mass spectrometry (see e.g. Bugni T S J. Nat. Prod., 2017, 80(2):574-575). All references are hereby incorporated by reference in their entirety.

The level of expression of CFHR4 may be measured using techniques described herein and/or well known in the art, as reviewed in, for example, Roth C M, Curr. Issues Mol. Biol. 2002 4:93-100 and Kukurba K R and Montgomery S B, Cold Spring Harb Protoc. 2015, (11):951-969, which are hereby incorporated by reference in their entirety. For example, gene expression can be measured using quantitative PCR, real-time PCT, sequencing techniques e.g. RNA-seq, next-generation sequencing, microarrays, Northern blot, and ribonuclease protection assay (RPA). One skilled in the art will be able to appreciate a suitable technique(s) for measuring expression of CFHR4, as required. In some cases, the total RNA or cDNA may be extracted and isolated first from a cell sample.

For standard molecular biology techniques, see Sambrook, J., Russel, D. W. Molecular Cloning, A Laboratory Manual. 3 ed. 2001, Cold Spring Harbor, N.Y.: Cold Spring Harbor Laboratory Press

Kits

In an aspect of the invention a kit of parts is provided, the kit comprising a container having a complement-targeted therapeutic and/or an agent that decreases the level of FHR-4 and/or decreases expression of a gene encoding FHR-4, and instructions for determining the level of FHR-4 and/or the level of expression of a gene encoding FHR-4 in one or more biological samples.

C3 and Complement Regulatory Proteins

Processing of C3 is described, for example, in Foley et al. J Thromb Haemostasis (2015) 13: 610-618, which is hereby incorporated by reference in its entirety. Human C3 (UniProt: P01024; SEQ ID NO:7) comprises a 1,663 amino acid sequence (including an N-terminal, 22 amino acid signal peptide). Amino acids 23 to 667 encode C3 β chain (SEQ ID NO:8), and amino acids 749 to 1,663 encode C3 α′ chain (SEQ ID NO:9). C3 β chain and C3 α′ chain associate through interchain disulphide bonds (formed between cysteine 559 of C3 β chain, and cysteine 816 of the C3 α′ chain) to form C3b.

Processing of C3b to the proteolytically-inactive form iC3b involves proteolytic cleavage of the C3b α′ chain at amino acid positions 1303 and 1320 to form an α′ chain fragment 1 (corresponding to amino acid positions 672 to 748 of C3), and an α′ chain fragment 2 (corresponding to amino acid positions 1321 to 1,663 of C3). Thus, iC3b comprises the C3 β chain, C3 a′ chain fragment 1 and C3 a′ chain fragment 2 (associated via disulphide bonds). Cleavage of the α′ chain also liberates C3f, which corresponds to amino acid positions 1304 to 1320 of C3.

Processing of C3b to iC3b is performed by Complement Factor I (encoded in humans by the gene CFI). Human Complement Factor I (UniProt: P05156) has a 583 amino acid sequence (including an N-terminal, 18 amino acid signal peptide). The precursor polypeptide is cleaved by furin to yield the mature Complement Factor I, comprising a heavy chain (amino acids 19 to 335), and light chain (amino acids 340 to 583) linked by interchain disulphide bonds. Amino acids 340 to 574 of the light chain encode the proteolytic domain of Complement Factor I which is a serine protease containing the catalytic triad responsible for cleaving C3b to produce iC3b (Ekdahl et al., J Immunol (1990) 144 (11): 4269-74).

Proteolytic cleavage of C3b by Complement Factor I to yield iC3b is facilitated by co-factors for Complement Factor I. Molecules capable of acting as co-factors for Complement Factor I include Complement Factor H, Complement Receptor 1 (CR1), CD46, CD55 and C4-binding protein (C4BP), SPICE, VCP (or VICE), and MOPICE.

Complement Factor H structure and function is reviewed e.g. in Wu et al., Nat Immunol (2009) 10(7): 728-733, which is hereby incorporated by reference in its entirety. Human Complement Factor H (UniProt: P08603) has a 1,233 amino acid sequence (including an N-terminal, 18 amino acid signal peptide), and comprises 20 complement control protein (CCP) domains of ˜60 amino acids.

Complement Receptor 1 (CR1) structure and function is reviewed e.g. in Khera and Das, Mol Immunol (2009) 46(5): 761-772 and Jacquet et al., J Immunol (2013) 190(7): 3721-3731, both of which are hereby incorporated by reference in their entirety. Human CR1 (UniProt: P17927) has a 2,039 amino acid sequence (including an N-terminal, 41 amino acid signal peptide), and comprises 30 complement control protein (CCP) domains.

CD46 (also referred to as Membrane Co-factor Protein (MCP)) structure and function is described e.g. in Liszewski and Atkinson, Human Genomics (2015) 9:7 and Liszewski et al., J Biol Chem (2000) 275: 37692-37701, both of which are hereby incorporated by reference in their entirety. Human CD46 (UniProt: P15529) has a 392 amino acid sequence (including an N-terminal, 34 amino acid signal peptide), and comprises a 309 amino acid extracellular domain (UniProt: P15529 positions 35 to 343), a 23 amino acid transmembrane domain (UniProt: P15529 positions 344 to 366), and a 26 amino acid cytoplasmic domain (UniProt: P15529 positions 367 to 392).

CD55 (also referred to as Decay Accelerating Factor (DAF)) structure and function is described e.g. Brodbeck et al., Immunology (2000) 101(1):104-111, which is hereby in incorporated by reference in its entirety. Human CD55 (UniProt: P08174) has a 381 amino acid sequence (including an N-terminal, 34 amino acid signal peptide), and comprises four CCP domains.

C4-binding protein (C4BP) structure and function is described in Blom et al., J Biol Chem (2001) 276(29): 27136-27144 and Fukui et al., J Biochem (2002) 132(5):719-728, both of which are hereby incorporated by reference in their entirety. Human C4BP (UniProt: P04003) has a 597 amino acid sequence (including an N-terminal, 48 amino acid signal peptide), and comprises 8 CCP domains.

Sequences

| SEQ ID | | |
|---|---|---|
| NO: | Description | Sequence |
| 1 | Human FHR-4A | MLLLINVILTLWVSCANGQEVKPCDFPEIQHGGLYYKSLRRLYFPAAAGQSY |
| | (isoform 1) | SYYCDQNFVTPSGSYWDYIHCTQDGWSPTVPCLRTCSKSDVEIENGFISES |
| | Uniprot: Q92496-1 | SSIYILNEETQYNCKPGYATAEGNSSGSITCLQNGWSTQPICIKFCDMPVFE |
| | Entry version 145 | NSRAKSNGMWFKLHDTLDYECYDGYESSYGNTTDSIVCGEDGWSHLPTC |
| | (28 Feb. 2018), | YNSSENCGPPPPISNGDTTSFPQKVYLPWSRVEYQCQSYYELQGSKYVTC |
| | Sequence version 3 | SNGDWSEPPRCISMKPCEFPEIQHGHLYYENTRRPYFPVATGQSYSYYCD |
| | (22 Jan. 2014) | QNFVTPSGSYWDYIHCTQDGWLPTVPCLRTCSKSDIEIENGFISESSSIYILN |
| | | KEIQYKCKPGYATADGNSSGSITCLQNGWSAQPICIKFCDMPVFENSRAKS |
| | | NGMRFKLHDTLDYECYDGYEISYGNTTGSIVCGEDGWSHFPTCYNSSEKC |
| | | GPPPPISNGDTTSFLLKVYVPQSRVEYQCQSYYELQGSNYVTCSNGEWSE |
| | | PPRCIHPCIITEENMNKNNIQLKGKSDIKYYAKTGDTIEFMCKLGYNANTSVL |
| | | SFQAVCREGIVEYPRCE |
| 2 | Human FHR-4A | MLLLINVILTLWVSCANGQVKPCDFPEIQHGGLYYKSLRRLYFPAAAGQSYS |
| | (isoform 2) | YYCDQNFVTPSGSYWDYIHCTQDGWSPTVPCLRTCSKSDVEIENGFISESS |
| | Uniprot: Q92496-2 | SIYILNEETQYNCKPGYATAEGNSSGSITCLQNGWSTQPICIKFCDMPVFEN |
| | Deletion at position | SRAKSNGMWFKLHDTLDYECYDGYESSYGNTTDSIVCGEDGWSHLPTCY |
| | 20 | NSSENCGPPPPISNGDTTSFPQKVYLPWSRVEYQCQSYYELQGSKYVTCS |
| | Entry version 145 | NGDWSEPPRCISMKPCEFPEIQHGHLYYENTRRPYFPVATGQSYSYYCDQ |
| | (28 Feb. 2018), | NFVTPSGSYWDYIHCTQDGWLPTVPCLRTCSKSDIEIENGFISESSSIYILNK |
| | Sequence version 3 | EIQYKCKPGYATADGNSSGSITCLQNGWSAQPICIKFCDMPVFENSRAKSN |
| | (22 Jan. 2014) | GMRFKLHDTLDYECYDGYEISYGNTTGSIVCGEDGWSHFPTCYNSSEKCG |
| | | PPPPISNGDTTSFLLKVYVPQSRVEYQCQSYYELQGSNYVTCSNGEWSEP |
| | | PRCIHPCIITEENMNKNNIQLKGKSDIKYYAKTGDTIEFMCKLGYNANTSVLS |
| | | FQAVCREGIVEYPRCE |
| 3 | Human FHR-4B | MLLLINVILTLWVSCANGQEVKPCDFPEIQHGGLYYKSLRRLYFPAAAGQSY |
| | Uniprot: Q92496-3 | SYYCDQNFVTPSGSYWDYIHCTQDGWSPTVPCLRTCSKSDIEIENGFISES |
| | Deletion at | SSIYILNKEIQYKCKPGYATADGNSSGSITCLQNGWSAQPICIKFCDMPVFE |
| | positions 80-326 of | NSRAKSNGMRFKLHDTLDYECYDGYEISYGNTTGSIVCGEDGWSHFPTCY |
| | FHR-4A | NSSEKCGPPPPISNGDTTSFLLKVYVPQSRVEYQCQSYYELQGSNYVTCS |
| | Entry version 145 | NGEWSEPPRCIHPCIITEENMNKNNIQLKGKSDIKYYAKTGDTIEFMCKLGY |
| | (28 Feb. 2018), | NANTSVLSFQAVCREGIVEYPRCE |
| | Sequence version 3 | |
| | (22 Jan. 2014) | |
| 4 | Human FHR-4 | MLLLINVILTLWVSCANGQ |
| | signal peptide | |
| | Positions 1-19 | |
| | Uniprot: Q92496-1 | |
| 5 | Human FHR-4A | EVKPCDFPEIQHGGLYYKSLRRLYFPAAAGQSYSYYCDQNFVTPSGSYWD |
| | mature protein | YIHCTQDGWSPTVPCLRTCSKSDVEIENGFISESSSIYILNEETQYNCKPGY |
| | Positions 20-578 | ATAEGNSSGSITCLQNGWSTQPICIKFCDMPVFENSRAKSNGMWFKLHDT |
| | Uniprot: Q92496-1 | LDYECYDGYESSYGNTTDSIVCGEDGWSHLPTCYNSSENCGPPPPISNGD |
| | Entry version 145 | TTSFPQKVYLPWSRVEYQCQSYYELQGSKYVTCSNGDWSEPPRCISMKP |
| | (28 Feb. 2018), | CEFPEIQHGHLYYENTRRPYFPVATGQSYSYYCDQNFVTPSGSYWDYIHC |
| | Sequence version 3 | TQDGWLPTVPCLRTSKSDIEIENGFISESSSIYILNKEIQYKCKPGYATADGN |
| | (22 Jan. 2014) | SSGSITCLQNGWSAQPICIKFCDMPVFENSRAKSNGMRFKLHDTLDYECYD |
| | | GYEISYGNTTGSIVCGEDGWSHFPTCYNSSEKCGPPPPISNGDTTSFLLKV |
| | | YVPQSRVEYQCQSYYELQGSNYVTCSNGEWSEPPRCIHPCIITEENMNKN |
| | | NIQLKGKSDIKYYAKTGDTIEFMCKLGYNANTSVLSFQAVCREGIVEYPRCE |
| 6 | Human FHR-4B | EVKPCDFPEIQHGGLYYKSLRRLYFPAAAGQSYSYYCDQNFVTPSGSYWD |
| | mature protein | YIHCTQDGWSPTVPCLRTCSKSDIEIENGFISESSSIYILNKEIQYKCKPGYAT |
| | Positions 20-331 | ADGNSSGSITCLQNGWSAQPICIKFCDMPVFENSRAKSNGMRFKLHDTLD |
| | Uniprot: Q92496-3 | YECYDGYEISYGNTTGSIVCGEDGWSHFPTCYNSSEKCGPPPPISNGDTTS |
| | | FLLKVYVPQSRVEYQCQSYYELQGSNYVTCSNGEWSEPPRCIHPCIITEEN |
| | | MNKNNIQLKGKSDIKYYAKTGDTIEFMCKLGYNANTSVLSFQAVCREGIVEY |
| | | PRCE |
| 7 | Human C3 | MGPTSGPSLLLLLLTHLPLALGSPMYSIITPNILRLESEETMVLEAHDAQGDV |
| | (UniProt: P01024; | PVTVTVHDFPGKKLVLSSEKTVLTPATNHMGNVTFTIPANREFKSEKGRNK |
| | Entry version 221 | FVTVQATFGTQVVEKVVLVSLQSGYLFIQTDKTIYTPGSTVLYRIFTVNHKLL |
| | (20 Dec. 2017); | PVGRTVMVNIENPEGIPVKQDSLSSQNQLGVLPLSWDIPELVNMGQWKIRA |
| | Sequence version 2 | YYENSPQQVFSTEFEVKEYVLPSFEVIVEPTEKFYYIYNEKGLEVTITARFLY |
| | (12 Dec. 2006)) | GKKVEGTAFVIFGIQDGEQRISLPESLKRIPIEDGSGEVVLSRKVLLDGVQNP |
| | including signal | RAEDLVGKSLYVSATVILHSGSDMVQAERSGIPIVTSPYQIHFTKTPKYFKP |
| | peptide | GMPFDLMVFVTNPDGSPAYRVPVAVQGEDTVQSLTQGDGVAKLSINTHPS |
| | | QKPLSITVRTKKQELSEAEQATRTMQALPYSTVGNSNNYLHLSVLRTELRP |
| | | GETLNVNFLLRMDRAHEAKIRYYTYLIMNKGRLLKAGRQVREPGQDLVVLP |
| | | LSITTDFIPSFRLVAYYTLIGASGQREVVADSVWVDVKDSCVGSLVVKSGQS |
| | | EDRQPVPGQQMTLKIEGDHGARVVLVAVDKGVFVLNKKNKLIQSKIWDVV |
| | | EKADIGCTPGSGKDYAGVFSDAGLTFTSSSGQQTAQRAELQCPQPAARRR |
| | | RSVQLTEKRMDKVGKYPKELRKCCEDGMRENPMRFSCQRRTRFISLGEA |
| | | CKKVFLDCCNYITELRRQHARASHLGLARSNLDEDIIAEENIVSRSEFPESW |
| | | LWNVEDLKEPPKNGISTKLMNIFLKDSITTWEILAVSMSDKKGICVADPFEVT |
| | | VMQDFFIDLRLPYSVVRNEQVEIRAVLYNYRQNQELKVRVELLHNPAFCSL |
| | | ATTKRRHQQTVTIPPKSSLSVPYVIVPLKTGLQEVEVKAAVYHHFISDGVRK |
| | | SLKVVPEGIRMNKTVAVRTLDPERLGREGVQKEDIPPADLSDQVPDTESET |
| | | RILLQGTPVAQMTEDAVDAERLKHLIVTPSGCGEQNMIGMTPTVIAVHYLDE |
| | | TEQWEKFGLEKRQGALELIKKGYTQQLAFRQPSSAFAAFVKRAPSTWLTA |
| | | YVVKVFSLAVNLIAIDSQVLCGAVKWLILEKQKPDGVFQEDAPVIHQEMIGG |
| | | LRNNNEKDMALTAFVLISLQEAKDICEEQVNSLPGSITKAGDFLEANYMNLQ |
| | | RSYTVAIAGYALAQMGRLKGPLLNKFLTTAKDKNRWEDPGKQLYNVEATS |
| | | YALLALLQLKDFDFVPPVVRWLNEQRYYGGGYGSTQATFMVFQALAQYQK |
| | | DAPDHQELNLDVSLQLPSRSSKITHRIHWESASLLRSEETKENEGFTVTAE |
| | | GKGQGTLSVVTMYHAKAKDQLTCNKFDLKVTIKPAPETEKRPQDAKNIMIL |
| | | EICTRYRGDQDATMSILDISMMTGFAPDTDDLKQLANGVDRYISKYELDKAF |
| | | SDRNTLIIYLDKVSHSEDDCLAFKVHQYFNVELIQPGAVKVYAYYNLEESCT |
| | | RFYHPEKEDGKLNKLCRDELCRCAEENCFIQKSDDKVTLEERLDKACEPGV |
| | | DYVYKTRLVKVQLSNDFDEYIMAIEQTIKSGSDEVQVGQQRTFISPIKCREA |
| | | LKLEEKKHYLMWGLSSDFWGEKPNLSYIIGKDTWVEHWPEEDECQDEEN |
| | | QKQCQDLGAFTESMVVFGCPN |
| 8 | Human C3 β chain | SPMYSIITPNILRLESEETMVLEAHDAQGDVPVTVTVHDFPGKKLVLSSEKT |
| | (UniProt: P01024; | VLTPATNHMGNVTFTIPANREFKSEKGRNKFVTVQATFGTQVVEKVVLVSL |
| | Entry version 221 | QSGYLFIQTDKTIYTPGSTVLYRIFTVNHKLLPVGRTVMVNIENPEGIPVKQD |
| | (20 Dec. 2017); | SLSSQNQLGVLPLSWDIPELVNMGQWKIRAYYENSPQQVFSTEFEVKEYVL |
| | Sequence version 2 | PSFEVIVEPTEKFYYIYNEKGLEVTITARFLYGKKVEGTAFVIFGIQDGEQRIS |
| | (12 Dec. 2006); | LPESLKRIPIEDGSGEVVLSRKVLLDGVQNPRAEDLVGKSLYVSATVILHSG |
| | residues 23-667) | SDMVQAERSGIPIVTSPYQIHFTKTPKYFKPGMPFDLMVFVTNPDGSPAYR |
| | | VPVAVQGEDTVQSLTQGDGVAKLSINTHPSQKPLSITVRTKKQELSEAEQA |
| | | TRTMQALPYSTVGNSNNYLHLSVLRTELRPGETLNVNFLLRMDRAHEAKIR |
| | | YYTYLIMNKGRLLKAGRQVREPGQDLVVLPLSITTDFIPSFRLVAYYTLIGAS |
| | | GQREVVADSVWVDVKDSCVGSLVVKSGQSEDRQPVPGQQMTLKIEGDHG |
| | | ARVVLVAVDKGVFVLNKKNKLIQSKIWDVVEKADIGCTPGSGKDYAGVFSD |
| | | AGLTFTSSSGQQTAQRAELQCPQPAA |
| 9 | Human C3 α′ chain | SNLDEDIIAEENIVSRSEFPESWLWNVEDLKEPPKNGISTKLMNIFLKDSITT |
| | (UniProt: P01024; | WEILAVSMSDKKGICVADPFEVIVMQDFFIDLRLPYSVVRNEQVEIRAVLYN |
| | Entry version 221 | YRQNQELKVRVELLHNPAFCSLATTKRRHQQTVTIPPKSSLSVPYVIVPLKT |
| | (20 Dec. 2017); | GLQEVEVKAAVYHHFISDGVRKSLKVVPEGIRMNKTVAVRTLDPERLGREG |
| | Sequence version 2 | VQKEDIPPADLSDQVPDTESETRILLQGTPVAQMTEDAVDAERLKHLIVTPS |
| | (12 Dec. 2006); | GCGEQNMIGMTPTVIAVHYLDETEQWEKFGLEKRQGALELIKKGYTQQLAF |
| | residues 749-1663) | RQPSSAFAAFVKRAPSTWLTAYVVKVFSLAVNLIAIDSQVLCGAVKWLILEK |
| | | QKPDGVFQEDAPVIHQEMIGGLRNNNEKDMALTAFVLISLQEAKDICEEQV |
| | | NSLPGSITKAGDFLEANYMNLQRSYTVAIAGYALAQMGRLKGPLLNKFLTTA |
| | | KDKNRWEDPGKQLYNVEATSYALLALLQLKDFDFVPPVVRWLNEQRYYGG |
| | | GYGSTQATFMVFQALAQYQKDAPDHQELNLDVSLQLPSRSSKITHRIHWE |
| | | SASLLRSEETKENEGFTVTAEGKGQGTLSVVTMYHAKAKDQLTCNKFDLK |
| | | VTIKPAPETEKRPQDAKNTMILEICTRYRGDQDATMSILDISMMTGFAPDTD |
| | | DLKQLANGVDRYISKYELDKAFSDRNTLIIYLDKVSHSEDDCLAFKVHQYFN |
| | | VELIQPGAVKVYAYYNLEESCTRFYHPEKEDGKLNKLCRDELCRCAEENCF |
| | | IQKSDDKVTLEERLDKACEPGVDYVYKTRLVKVQLSNDFDEYIMAIEQTIKS |
| | | GSDEVQVGQQRTFISPIKCREALKLEEKKHYLMWGLSSDFWGEKPNLSYII |
| | | GKDTWVEHWPEEDECQDEENQKQCQDLGAFTESMVVFGCPN |
| 10 | Construct FHR4 #1 | GAATCACACTTGGTAACTAAT |
| | Position: 46 | |
| | siRNA target | |
| 11 | Construct FHR4 #1 | GCCAAGAATGTACCTATTATA |
| | Position: 46 | |
| | siRNA scrambled | |
| 12 | Construct FHR4 #2 | GGTCAAGAGTCGAGTACCAGT |
| | Position: 835 | |
| | siRNA target | |
| 13 | Construct FHR4 #2 | GGGTGAATCCCGCATGGTAAA |
| | Position: 835 | |
| | siRNA scrambled | |
| | (negative control) | |
| 14 | Construct FHR4 #3 | GAATGCTACGATGGATATGAA |
| | Position: 1401 | |
| | siRNA target | |
| 15 | Construct FHR4 #3 | GATAAGGTGAGCCATTAGATA |
| | Position: 1401 | |
| | siRNA scrambled | |
| | (negative control) | |
| 16 | Construct FHR4 #4 | GGAACCACCAAGATGCATACA |
| | Position: 1658 | |
| | siRNA target | |
| 17 | Construct FHR4 #4 | GCACTCAAGAAGACGATACCA |
| | Position: 1658 | |
| | siRNA scrambled | |
| | (negative control) | |
| 18 | Construct FHR4 #5 | GGATATAATGCGAATACATCA |
| | Position: 1794 | |
| | siRNA target | |
| 19 | Construct FHR4 #5 | GCATAAGAATATACGTTCAGA |
| | Position: 1794 | |
| | siRNA scrambled | |
| | (negative control) | |
| 20 | Construct FHR4 #6 | GCATATTGTACAGTATACCTA |
| | Position: 2154 | |
| | siRNA target | |
| 21 | Construct FHR4 #6 | GTTGCTATCGTCTATAAACAA |
| | Position: 2154 | |
| | siRNA scrambled | |
| | (negative control) | |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

Aspects and embodiments of the present invention will now be discussed with reference to the accompanying figures. Further aspects and embodiments will be apparent to those skilled in the art. All documents mentioned in this text are incorporated herein by reference.

### SUMMARY OF THE FIGURES

Embodiments and experiments illustrating the principles of the invention will now be discussed with reference to the accompanying figures.

FIGS. 1A and 1B. Graphs showing gene expression analysis of CFHR4. (A) Gene expression analysis of CFHR4 and CFH in eye tissue from AMD and non-AMD subjects. Gene expression was measured against normalised expression levels. (B) Gene expression analysis of CFHR4 in 27 human tissues.

FIGS. 2A and 2B. Images of FHR-4 protein in eye tissues detected by immunohistochemistry. Scale bars equal 20 μm. (A) Image showing FHR-4 localisation in intercapillary septa of choriocapillaris and BrM. (B) image showing FHR-4 localisation in a druse.

FIG. 3. Surface plasmon resonance (SPR) sensorgram of FHR-4 binding to immobilised C3b.

FIG. 4. Graph showing solid phase binding assays in which increasing concentrations of FHR-4 are able to out-compete the binding between C3b and FH/FHL-1.

FIG. 5. Chart illustrating the effect of FHR-4 on the activity of FHL-1 as a co-factor for FI, measured by C3b breakdown.

FIG. 6. Schematic showing the proposed role of FHR-4 in C3 convertase formation.

FIGS. 7A to 7C. Graphs showing FHR-4 levels in AMD and non-AMD subjects. (A) FHR-4 concentration in plasma of 187 subjects (‘discovery cohort’) with either advanced AMD or no AMD. (B) Analysis of percentage distribution of plasma FHR-4 concentrations from the discovery cohort. (C) Analysis of percentage distribution of plasma FHR-4 concentrations from 518 subjects (‘full cohort’) with either AMD or no AMD.

FIGS. 8A and 8B. Graphs showing expression of CFHR4 after siRNA knockdown in HuH liver cells. (A) Expression of CFHR4 after transfection with siRNAs 1-6, individual or pooled, or their equivalent siRNA negative controls (scrambled siRNA). Expression of CFHR4 is normalised relative to GAPDH expression. (B) Percentage expression of CFHR4 after transfection with siRNAs 1-6, individual or pooled, with respect to siRNA negative controls (scrambled siRNA).

### EXAMPLES

### Example 1

CFHR4 Gene Expression

Eye tissue taken from AMD and non-AMD subjects was analysed for expression of the gene encoding FHR-4 (CFHR4).

CFHR4 expression was analysed by the inventors using publically-available data generated by Whitmore S S et al., Altered gene expression in dry age-related macular degeneration suggests early loss of choroidal endothelial cells. Mol Vis 2013; 19:2274-97. Briefly, Whitmore et al. dissected donor eyes and separated the macular RPE and choroid from the retina. RNA was extracted from the RPE and choroids and expression of over 10,000 genes was measured using exon-based microarrays (Affymetrix).

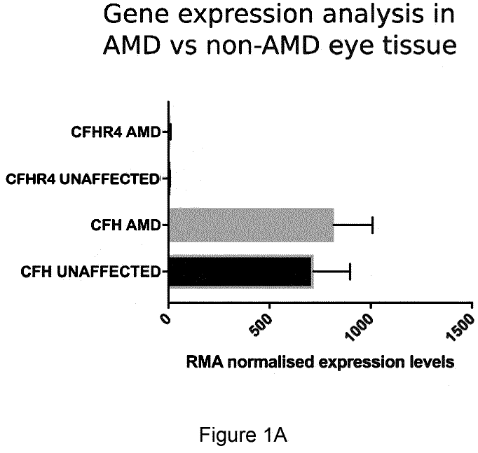

The results of the inventors' analysis are shown in FIG. 1A. CFHR4 was found not to be expressed in the retina or choroid of donor eyes from either AMD or non-AMD subjects. In contrast, CFH (encoding the FH protein) was found to be expressed in both cohorts.

Next, a variety of human tissues were analysed for CFHR4 gene expression.

Tissue-specific CFHR4 expression was analysed by the inventors using publically-available data generated as part of the Genotype-Tissue Expression (GTEx) project, see GTEx Consortium (2015) Human Genomics. The genotype-tissue expression (GTEx) pilot analysis: multitissue gene regulation in humans. Science, 348, 648-660.

The inventors' analysis of CFHR4 gene expression in 27 tissues is shown in FIG. 1B. Significant CFHR4 gene expression was only detected in liver tissue.

Localisation of FHR-4 Protein

The localisation of FHR-4 protein in eye tissue was analysed by immunohistochemistry.

Tissue sections (10 μm) were stained for the presence of endogenous FHR-4, collagen IV or C3/C3b using methods described previously (Clark S J et al., J Immunol. 2014, 193: 4962-4970). Briefly, tissue sections were incubated with chilled (−20° C.) histological grade acetone (Sigma-Aldrich) and methanol (mixed 1:1) for 20 seconds before thorough washing with PBS. Tissue sections were blocked with 0.1% (w/v) BSA, 1% (v/v) goat serum, and 0.1% (v/v) Triton X-100 in PBS for 1 hat room temperature. After washing with PBS, tissue sections were incubated with an antibody combination of 10 μg/ml of anti-FHR-4 (see below) mixed with 1 μg/ml Collagen IV rabbit polyclonal antibody for 16 h at 4° C. Sections were washed and biotinylated anti-mouse IgG (Catalogue No. BA_9200, Vector laboratories, Inc) diluted 1:250 in PBS was applied for 1 hour to amplify the FHR-4 signal. Slides were subsequently washed and Alexa Fluor® 647 streptavidin (catalogue no: S32357, Invitrogen) diluted 1:250 in PBS and Alexa Fluor®488-conjugated goat anti-rabbit Ab (Invitrogen, USA) diluted 1:500 in PBS were added for 2 h at room temperature. After washing DAPI was applied as a nuclear counterstain (at 0.3 mM for 5 min) prior to mounting with medium (Vectashield; H-1400, Vector Laboratories, Peterborough, UK) and application of a coverslip.

In the case of blank control sections, the exact same protocol was followed but PBS replaced the primary antibody. To test antibody specificity in immunohistochemistry pre-adsorption experiments were performed whereby 10-fold molar excess of pure recombinant FHR-4 is premixed with the anti-FHR-4 Ab prior to application to the tissue sections. In all cases images were collected on a Zeiss Axioimager.D2 upright microscope using a 40×/0.5 EC Plan-neofluar and 100×/0.5 EC Plan-neofluar objective and captured using a Coolsnap HQ2 camera (Photometrics) through Micromanager software v1.4.23. Specific band pass filter sets for DAPI, FITC and Cy5 were used to prevent bleed through from one channel to the next. Images were then processed and analysed using Fiji ImageJ (http://imagej.net/Fiji/Downloads). To prevent bleed-through of color from one channel to the next, specific band pass filter sets were used for DAPI, FITC, and Cy-5. All images were handled using ImageJ64 (version 1.40g; http://rsb.info.nih.gov/ij).

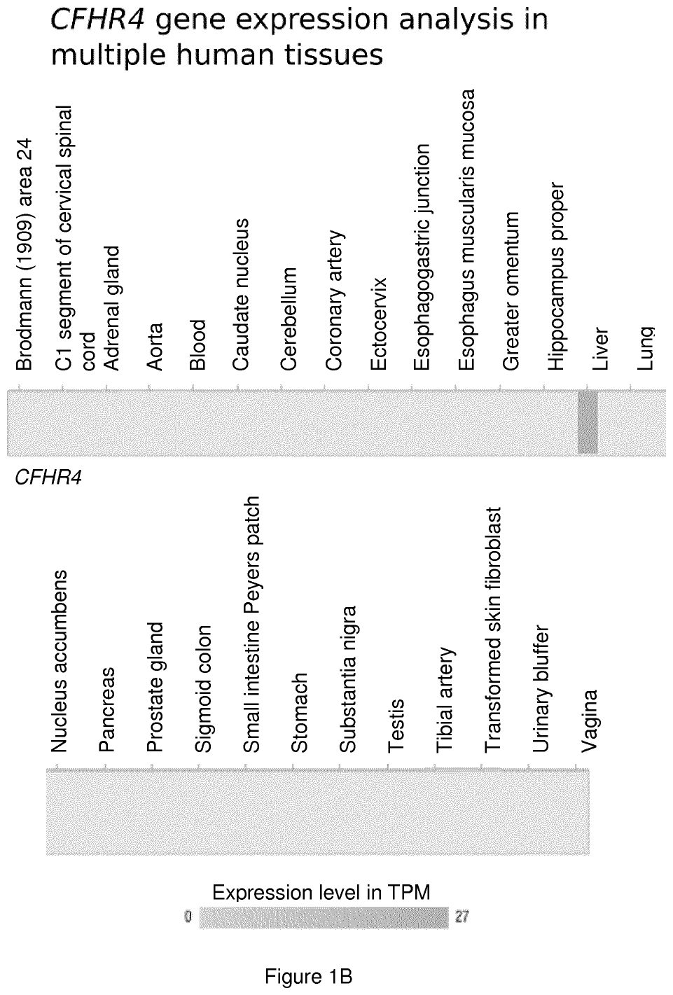

The results are shown in FIGS. 2A and 2B. FIG. 2A shows that FHR-4 localises to the intercapillary septa and that weak labelling is also observed in the BrM. FIG. 2B shows FHR-4 localised in a druse. Scale bars equal 20 μm.

Thus, FHR-4 is synthesised in the liver before accumulating in tissues near the eye.

### Example 2

### Generation of FHR-4 Antibody

Mice are immunised with recombinant FHR-4 in complete Freund's adjuvant, using standard protocols known in the art. The titre of anti-FHR-4 antibody is assessed by screening sera from individual mice in a capture ELISA. Spleen cells are harvested and fused with myeloma cells to generate hybridomas, using standard protocols. Hybridomas are selected, left to grow, and then screened for antibody production. Positive cells are expanded and antibodies are purified.

### Example 3

FHR-4 Binds to C3b

The ability of FHR-4 to bind to immobilised C3b was analysed using surface plasmon resonance (SPR).

SPR was performed using a Biacore 3000 (GE Healthcare). The sensor surfaces were prepared by immobilizing human C3b onto the flow cells of a Biacore series S carboxymethylated dextran (CM5) sensor chip (GE Healthcare) using standard amine coupling and included blank flow cells were no C3b protein was present. Experiments were performed at 25° C. and a flow rate of 15 μl/min in PBS with 0.05% surfactant P20. FHR-4 was injected in triplicate at concentration ranging from 1 to 100 μg/ml. Samples were injected for 150 seconds and dissociated for another 200 seconds and the chip was regenerated between injections with 1M NaCl for 1 min before chip is re-equilibrated into PBS with 0.05% surfactant P20 prior to the next injection. After subtraction of each response value from the blank cell, association and dissociation rate constants were determined by global data analysis. All curves were fitted using a 1:1 Langmuir association/dissociation model (BIAevaluation 4.1; GE Healthcare).

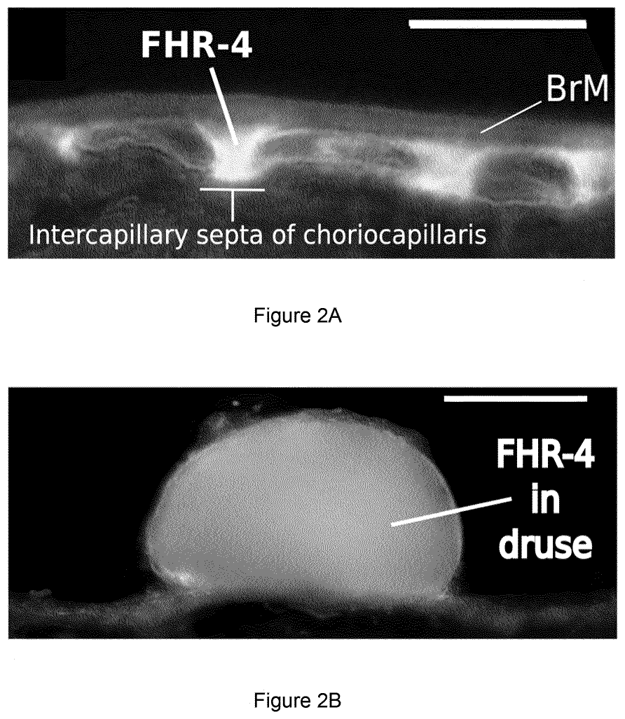

The results are shown in FIG. 3, in which FHR-4 is demonstrated to bind to immobilised C3b with a KD of 1.1×10⁻⁶ M.

FHR-4 Competes for C3b Binding with FH/FHL-1

Solid phase binding assays were used to assess whether FHR-4 can out-compete FH and FHL-1 binding to C3b.

Purified C3b was adsorbed onto the wells of microtiter plates (Nunc Maxisorb, Kastrup, Denmark) at 1 μg/well in 100 μl/well PBS for 16 h at room temperature. Plates were blocked for 90 minutes at 37° C. with 300 μl/well 1% (w/v) BSA in assay buffer (20 mM HEPES, 130 mM NaCl, 0.05% (v/v) Tween-20, pH 7.3). This standard assay buffer (SAB) was used for all subsequent incubations, dilutions and washes and all steps were performed at room temperature. A constant concentration of 100 nM was made for either FH or FHL-1 in SAB and increasing concentrations of FHR-4 were used as a competitor, up to 500 nM. FH/FHR-4 and FHL-1/FHR-4 mixes were incubated with the immobilized C3b for 4 hours. After washing, bound FH or FHL-1 protein was detected by the addition of 100 μl/well of 0.5 μg/ml OX23 antibody and incubated for 30 minutes followed by washing and a 30-minute incubation in 100 μl of a 1:1000 dilution of AP-conjugated anti-mouse IgG (Sigma-Aldrich). Plates were developed using 100 μl/well of a 1 mg/ml disodium p-nitrophenylphosphate solution (Sigma-Aldrich) in 0.05 M Tris-HCl, 0.1 M NaCl, pH 9.3. The absorbance values at 405 nm were determined after 10 minutes of development at room temperature and corrected against blank wells (i.e., those with no immobilized C3b).

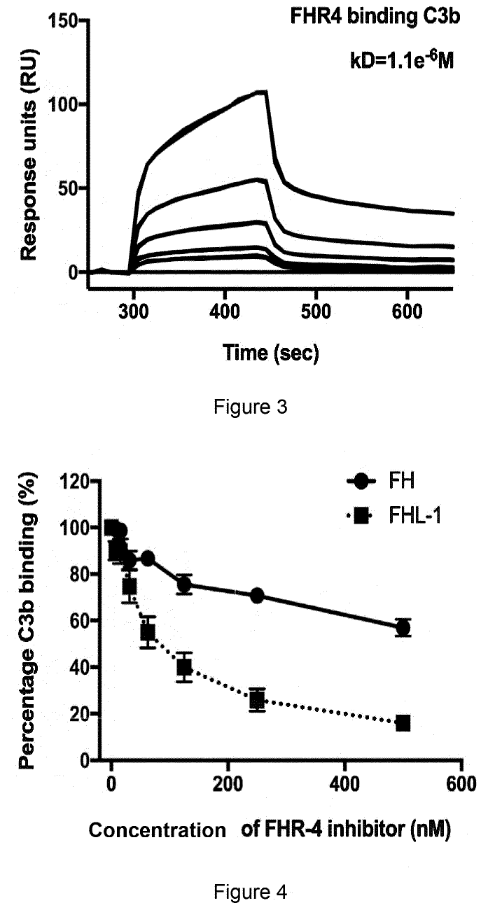

The results are shown in FIG. 4. Increasing concentrations of FHR-4 can progressively out-compete FH and FHL-1 binding to immobilised C3b.

Inhibition of C3b Breakdown by FHR-4

The ability of FHR-4 to inhibit the breakdown of C3b into iC3b was assessed.

The fluid phase cofactor activity of FHL-1 was measured by incubating purified FHL-1, C3b and FI together in a total volume of 20 μl PBS for 15 minutes at 37° C. For each reaction 2 μg C3b and 0.04 μg FI (Factor I) were used with varying concentrations of FHL-1 ranging from 0.015 μg to 1 μg per reaction. The assay was stopped with the addition of 5 μl 5×SDS reducing sample buffer and boiling for 10 minutes at 100° C. Samples were run on a 4-12% NuPAGE Bis Tris gel at 200V for 60 minutes in order to maximise the separation of the C3b breakdown product bands. Molecular weight markers used were Novex Sharp pre-stained protein standards (3.5-260 kDa, Cat. No. LC5800, Life Technologies, Paisley, UK). The density of the 68 kDa iC3b product band was measured using ImageJ64 (version 1.40g; rsb.info.nih.gov/ij) and used to track C3b breakdown efficiency of the FHL-1 proteins. For FHR-4 inhibition assays, the amount of FHL-1 used in the reaction was fixed at 1 μg and increasing amounts of FHR-4 were added to create up to a 5-fold molar excess of FHR-4 over FHL-1. Otherwise the reactions are performed under the same condition as previously. In all cases averaged data from three separate experiments were used.

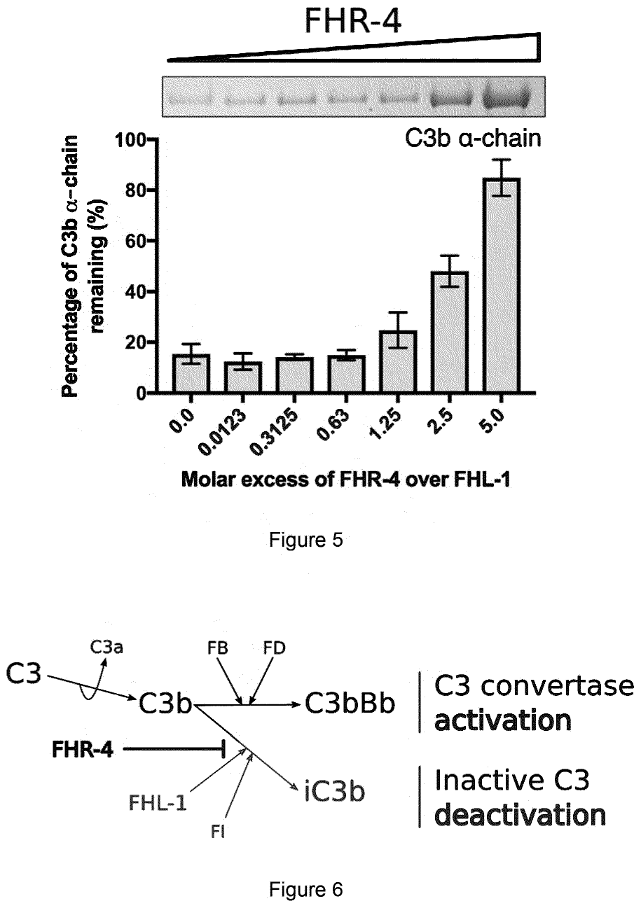

The results are shown in FIG. 5. Increasing concentrations of FHR-4 can progressively inhibit the breakdown of C3b into iC3b by FI and FHL-1.

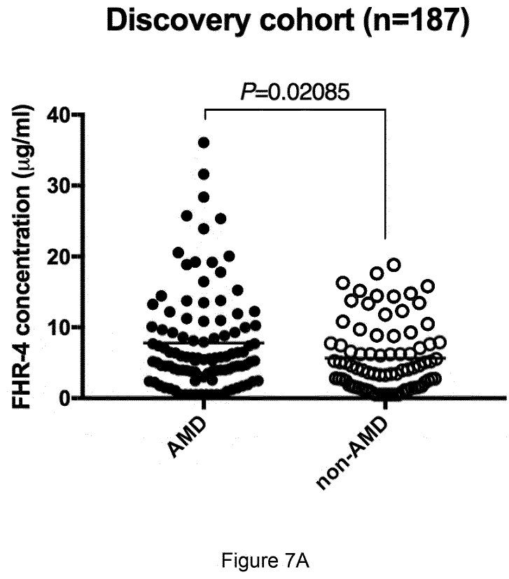

A schematic of the role of FHR-4 is shown in FIG. 6. FHR-4 prevents FHL-1 acting as a cofactor for factor I which causes failure of C3b inactivation (C3b is not converted into iC3b). This promotes C3 convertase formation and complement activation. FHR-4 thus promotes complement dysregulation.

### Example 4

Association Between FHR-4 Levels and Risk of AMD

FHR-4 levels were measured in blood plasma from patients with or without AMD to assess whether FHR-4 levels are associated with AMD risk.

Two separate AMD cohorts were analysed for FHR-4 levels: 187 plasma samples (‘discovery cohort’) from patients with advanced AMD compared to matched controls without AMD (106 AMD vs 81 non-AMD), and a ‘full cohort’ comprising 518 samples (304 AMD vs 214 non-AMD).

The samples were taken from a ‘Cambridge’ AMD study, a case-control study of AMD with participants recruited from ophthalmic clinics in London, the southeast of England, and the northwest of England between 2001 and 2007 (Yates et al., N Engl J Med. 2007, 357: 553-561). All patients had at least one eye affected by choroidal neovascularization (CNV) and/or geographic atrophy (GA). Patients were excluded if they had greater than 6 diopters of myopic refractive error or evidence of other inflammatory or retinovascular disease (such as retinal vessel occlusion, diabetic retinopathy, or chorioretinitis) that could contribute to the development of or confound the diagnosis of maculopathy. Controls were spouses, partners or friends of index patients. All participants described their race/ethnicity as white on a recruitment questionnaire. Participants were examined by an ophthalmologist and underwent color stereoscopic fundus photography of the macular region. Images were graded at the Reading Centre, Moorfields Eye Hospital, London, using the International Classification of Age-related Maculopathy and Macular Degeneration (Bird et al., The International ARM Epidemiological Study Group. Surv Ophthalmol. 1995, 39: 367-374). Blood samples were obtained at the time of interview and lithium-heparin plasma samples stored at −80° C. were later used for FHR-4 measurements.

FHR-4 concentrations were measured using an optimised in-house sandwich ELISA assay. Nunc-Immuno™ MaxiSorp™ 96-well plates were coated with 50 μl/well of monoclonal anti-FHR-4 antibody 4E9 at 5 μg/ml (in 0.1M carbonate buffer pH 9.6). After blocking in 2% BSA in PBS+0.1% Tween-20 (PBST), plates were washed in PBST and a dilution series of purified FHR-4 protein diluted in 0.1% PBST added to wells in duplicate to generate a standard curve. Test samples were added (50 μl/well) in duplicate at a 1:40 dilution to the remaining wells, and plates were incubated at 37° C. for 1.5 hours. Plates were washed in PBST, 50 μl/well of 1 μg/ml of HRP-labelled anti-FHR-4 monoclonal antibody was added and the plates were incubated for 1 hour at room temperature. After washing, 50 μl/well of orthophenylenediamine (SIGMAFAST™ OPD, Sigma-Aldrich, UK) was added to develop the plates and the reaction was stopped after 5 minutes by adding an equal volume of 10% sulphuric acid. Absorbance was measured in a plate reader at 492 nm and protein concentrations were interpolated from the standard curve plotted using GraphPadPrism5.

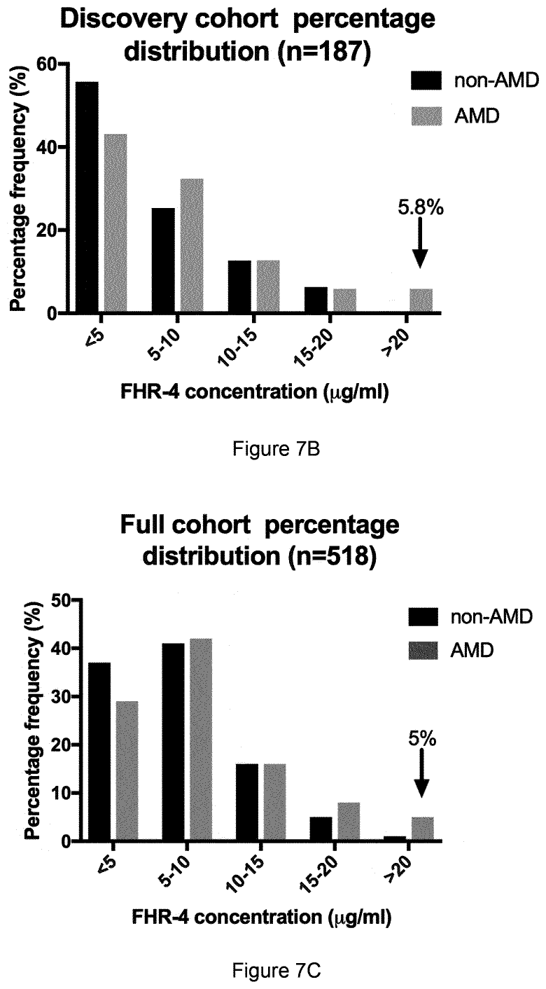

The results are shown in FIGS. 7A to 7C. (7A) FHR-4 measurements of plasma samples in the discovery cohort showed elevated mean FHR-4 levels in subjects with advanced AMD (7.8±0.7 μg/ml vs 5.7±0.5 μg/ml, P=0.0208). (7B) When the FHR-4 measurements from the discovery cohort were analysed for the percentage distribution of FHR-4 concentration, ˜12% of AMD subjects had blood plasma FHR-4 levels of greater than 15 μg/ml. Furthermore, 5.8% of AMD subjects had blood plasma FHR-4 levels of greater than 20 μg/ml, compared to 0% of non-AMD subjects. (7C) A replication experiment with the larger full cohort showed similar results, with 5% of AMD subjects having blood plasma FHR-4 levels of greater than 20 μg/ml.

### Example 5

siRNA Knockdown of CFHR4 Expression

siRNA molecules targeting different regions of CFHR4 were tested for their effect on CFHR4 expression in liver cells.

HuH cells from a human liver carcinoma cell line were cultured in Dulbecco's Modified Eagle's Medium with low glucose (DMEM, Sigma, catalogue number D6046) supplemented with 10% fetal fovine serum (FBS, Sigma, catalogue number F9665) and 1% penicillin streptomycin (Pen/Strep, Sigma P0781) in 5% CO2 incubator at 370C. The six siRNA target sequences (siRNA1-6; SEQ ID NO:10, 12, 14, 16, 18 and 20) and the six negative control siRNA sequences (scrambled siRNAs; SEQ ID NO:11, 13, 15, 17, 19 and 21) were designed in-house and were made by Ambion (Life technologies Ltd).

The human liver carcinoma cells were seeded in 24 well plates (50,000 cells/well) and cultured. After 24 hrs, the cells were transfected with either 10 nM of siRNA1-6, or all 6 siRNAs pooled together, or their corresponding scrambled siRNA control using 1 μl of Lipofectamine RNAimax (Invitrogen, catalogue number 13778-075) for 24 hours. All reactions were carried out in duplicates.

After 24 hours post-transfection, RNA was extracted using the Isolate RNA Mini Kit (Bioline, catalogue number BIO-52072) and cDNA was synthesised using the High-Capacity cDNA Reverse Transcription Kit (Applied Biosystems, catalogue number 4368814).

Quantitative PCR reactions were performed using pre-designed FAM-labeled TaqMan probes (Thermo Fisher Scientific, Life Technologies) following the manufacturer's instructions. In brief, 10 ng of cDNA was resuspended in a reaction mix including 0.5 ul of either CFHR4 TaqMan probe (H500198577_m1) or GAPDH TaqMan probe (Hs02758991_g1), 5 ul of 2× reaction mastermix (4440040), in a final reaction volume of 10 μl. Samples were run in duplicate using an ABI Step One thermocycler (Applied Biosystems) using the following thermal cycling conditions: 42° C. for 5 minutes, 95° C. for 10 seconds and 40 cycles of 95° C. for 5 seconds and 60° C. for 34 seconds. CFHR4 gene expression was normalised to GAPDH expression and relative expression determined by the ΔΔCt method.

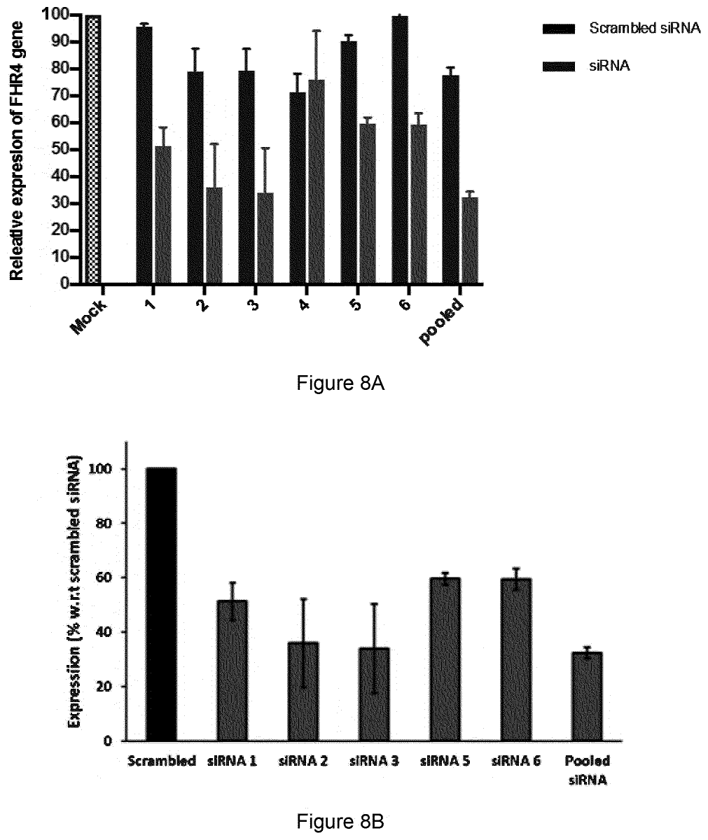

The results are shown in FIGS. 8A and 8B. The qPCR data demonstrate that siRNA 1-3, 5 and 6 (SEQ ID NO:10, 12, 14, 18, 20) all significantly reduced CFHR4 expression (8A; data for each siRNA are shown next to their equivalent scrambled siRNA negative control). siRNA 2 and 3 (SEQ ID NO:12, 14) and the pooled siRNA had the greatest CFHR4 knockdown effects. FIG. 8B shows that scrambled siRNA had no effect on CFHR4 expression.

1. A method for determining whether a subject is at risk of developing a complement-related disorder, the method comprising determining the level of FHR-4 in the blood of said subject.

2. The method according to claim 1, comprising determining an increase in the level of FHR-4 in the blood of the subject.

3. The method according to claim 1 or claim 2, wherein an increased level of FHR-4 indicates an increased risk of developing a complement-related disorder.

4. The method according to any one of claims 1 to 3, wherein the method comprises measuring the concentration of FHR-4 protein in the blood of said subject.

5. The method according to claim 4, wherein a FHR-4 concentration of >15 μg/ml indicates a high risk of said subject developing said disorder.

6. The method according to any one of claims 1 to 5, wherein the level and/or concentration of FHR-4 is determined in a blood-derived sample from the subject.

7. The method according to any one of claims 1 to 6, wherein the level and/or concentration of FHR-4 is determined in vitro.

8. The method according to any one of claims 1 to 7, wherein the disorder is selected from macular degeneration, age-related macular degeneration (AMD), geographic atrophy (dry′ (i.e. non-exudative) AMD), early AMD, intermediate AMD, late/advanced AMD, ‘wet’ (neovascular or exudative) AMD, choroidal neovascularisation (CNV), early-onset macular degeneration (EOMD), macular dystrophy, glaucoma, diabetic retinopathy, Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease.

9. The method according to claim 8, wherein the method further comprises determining in the subject the presence or absence of one or more genetic factors associated with AMD and/or EOMD and/or a macular dystrophy.

10. A complement-targeted therapeutic for use in a method of treating or preventing a complement-related disorder in a subject, wherein the subject has an increased level of FHR-4 and/or an increased level of a gene encoding FHR-4.

11. A method for treating or preventing a complement-related disorder in a subject, the method comprising administering an effective amount of a complement-targeted therapeutic to the subject, wherein the subject to be treated has an increased level of FHR-4 and/or an increased level of expression of a gene encoding FHR-4.

12. The complement-targeted therapeutic for use according to claim 10, or the method according to claim 11, wherein the subject has been determined to have an increased level of FHR-4 and/or an increased level of expression of a gene encoding FHR-4.

13. An agent that decreases the level of FHR-4 and/or decreases expression of a gene encoding FHR-4 for use in a method of treating or preventing a complement-related disorder in a subject, wherein the subject has an increased level of FHR-4 and/or an increased level of expression of a gene encoding FHR-4.

14. A method for treating or preventing a complement-related disorder in a subject, the method comprising administering an effective amount of an agent that decreases the level of FHR-4 and/or decreases expression of a gene encoding FHR-4 to the subject, wherein the subject has an increased level of FHR-4 and/or an increased level of expression of a gene encoding FHR-4.

15. The agent for use according to claim 13, or the method according to claim 14, wherein the subject has been determined to have an increased level of FHR-4 and/or an increased level of expression of a gene encoding FHR-4.

16. The agent for use according to claim 13 or claim 15, or the method according to claim 14 or claim 15, wherein the agent possesses one or more of the following properties: inhibits expression of the CFHR4 gene, degrades FHR-4 mRNA, binds to FHR-4 protein, sequesters FHR-4 protein, sequesters FHR-4 protein in the blood, competes for binding of FHR-4 protein, blocks activity of FHR-4 protein, reduces the concentration of FHR-4 in the blood, reduces the ability of FHR-4 protein to leave the blood, reduces the ability of FHR-4 protein to reach the eye, reduces the amount of FHR-4 in the eye, reduces the ability of FHR-4 protein to enter BrM, inhibits FHR-4-mediated signalling, modulates a reaction involving C3b, modulates a reaction involving FHR-4 and C3b, reduces the ability of FHR-4 protein to bind to C3b, competes with FHR-4 protein for C3b binding, encourages dissociation of FHR-4 from C3b, reduces C3 convertase activation, reduces production of C3bBb, increases C3 deactivation, increases production of iC3b, decreases complement activation, and/or inactivates a complement pathway.

17. The agent for use according to any one of claim 13, 15 or 16, or the method according to any one of claims 14 to 16, wherein the agent that decreases the amount of FHR-4 and/or decreases expression of a gene encoding FHR-4 is selected from: antisense nucleic acid, aptamer, antigen binding molecule, sequestering agent, and/or decoy receptor.

18. The agent for use according to any one of claims 10, 12, 13, or 15 to 17, or the method according to any one of claims 11, 12, or 14 to 17, wherein the complement-related disorder is selected from macular degeneration, age-related macular degeneration (AMD), geographic atrophy (dry′ (i.e. non-exudative) AMD), early AMD, intermediate AMD, late/advanced AMD, ‘wet’ (neovascular or exudative) AMD, choroidal neovascularisation (CNV), early-onset macular degeneration (EOMD), macular dystrophy, glaucoma, diabetic retinopathy, Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease.

19. A method for selecting a subject for treatment with a complement-targeted therapeutic or an agent that decreases the level of FHR-4 and/or decreases expression of a gene encoding FHR-4 to the subject, the method comprising determining the level of FHR-4 and/or the level of expression of a gene encoding FHR-4 in the subject and, optionally, where the level of FHR-4 and/or the level of expression of a gene encoding FHR-4 is increased, selecting the subject for treatment with the therapeutic or agent.

20. An agent that decreases the level of FHR-4 and/or decreases expression of a gene encoding FHR-4 for use in a method of treating or preventing an age-related macular degeneration (AMD) or early-onset macular degeneration (EOMD) or a macular dystrophy in a subject.

21. The agent for use according to claim 20, wherein the AMD is selected from geographic atrophy (dry′ (i.e. non-exudative) AMD), early AMD, intermediate AMD, late/advanced AMD, ‘wet’ (neovascular or exudative) AMD, and choroidal neovascularisation (CNV).

22. The agent for use according to claim 20 or claim 21, wherein the agent that decreases the amount of FHR-4 and/or decreases expression of a gene encoding FHR-4 is a sequestering agent and/or decoy receptor for FHR-4.
